(19) United States
(12) Patent Application Publication
(10) Pub. No.: US 2003/0148318 A1
(43) Pub. Date: Aug. 7, 2003

(54) Purified human polynucleotidic sequence hsCAS1, polypeptides encoded by this gene, necessary for the O-acetylation of the sialic acids and use as tools for the diagnosis and the prognosis of cancer diseases

(76) Inventors: Guilhem Janbon, Paris (FR); Frederique Provost, Boulogne (FR); Francoise Dromer, Paris (FR)

Correspondence Address: FINNEGAN, HENDERSON, FARABOW,, GARRETT & DUNNER, L.L.P., 1300 I. Street, NW, Washington, DC, 20005-3315, US

(21) Appl. No.: 10/214,390

(22) Filed: Aug. 8, 2002

Related U.S. Application Data
(60) Provisional application No. 60/314,098, filed on Aug. 23, 2001.

(51) Int. Cl. C12Q 1/68; C07H 21/04; C12N 9/10; C12P 21/02; C12N 5/06

(52) U.S. Cl. 435/6; 435/69.1; 435/193; 435/320.1; 435/325; 536/23.2; 536/24.3

(57) ABSTRACT

A human gene, designated hsCAS1 (2880 bp), encodes for a protein with a glycosyltransferase activity on sialic acids residues. The gene and the fragment thereof, as well as the proteins and polypeptides they encodes, are useful in diagnostic compositions and as a tools for the prognostic of the evolutions of several cancers diseases. This invention is also related on the Cryptococcus neoformans gene cnCAS1 and polypeptides encoded by this polynucleotide sequence.

### CROSS-REFERENCE TO RELATED APPLICATIONS

This application is based on and claims the benefit of U.S. Provisional Application No. 60/314,098, filed Aug. 23, 2001, (Attorney Docket No. 3495.6070) The entire disclosure of this application is relied upon and incorporated by reference herein.

### FIELD OF THE INVENTION

The present invention pertains to purified polypeptides, nucleics acids encoding such polypeptides, compositions comprising them, and their use as oligonucleotide primers or probes for the diagnosis and the prognosis of cancers in humans. The present invention is also directed to the sequence of hsCAS1 gene and it use as a diagnostic and/or prognostic tool of the evolution of several cancers by the studies of the different kinds of o-acetylation of sialic acids during the development and the differentiation of the cells and the use of such nucleic acids in diagnostic methods, kits, vaccines and therapy.

### BACKGROUND OF THE INVENTION

Cryptococcus neoformans is an opportunistic fungal pathogen that infects mainly immunocompromised patients, causing life-threatening infections of the central nervous system. This basidiomycete yeast exists in three varieties: C. neoformans var. grubii (serotype A) (Franzot et al., 1999) with a worldwide distribution, C. neoformans var. neoformans (serotype D), more frequently found in Europe; and C. neoformans var. gattii (serotypes B and C) limited to tropical and subtropical regions (Kwon-Chung and Bennett, 1984). C. neoformans cells have a thick extracellular polysaccharide capsule, which is one of the most important virulence factors along with the production of melanin (Casadevall and Perfect, 1998).

The capsule polysaccharides are composed of glucuronoxylomannans (GXM, 88% of its mass), galactoxylomannans (GalXM) and mannoproteins (MP) (Cherniak and Sundstrom, 1994). The GXM have been well characterized chemically and are the antigenic basis for serotype specificity. They consist of a (1→3)-α-D-mannopyranan backbone bearing β-D-xylopyranosyl, β-D-glucopyranosyluronic acid and 6-O-acetyl substituents. Variations of glucuronic acid and xylose substitution are used to characterize different chemotypes (Cherniak et al., 1998). The capsule protects C. neoformans cells against phagocytosis, alters antigen presentation, affects the production of cytokines, can be responsible for complement depletion in the host and inhibits leukocyte migration into infected sites (for review see (Buchanan and Murphy, 1998). Intravenous injection of capsule polysaccharide prior to infection with C. neoformans shortens the surv.ival of treated compared to control mice (Bennett and Hasenclever, 1965).

Recently, Chang et al. cloned four capsule-associated genes, CAP10, CAP59, CAP60 and CAP64, and showed that the specific deletion of any one was associated with loss of virulence and an acapsular phenotype (Chang and Kwon-Chung, 1994; Chang and Kwon-Chung, 1998; Chang and Kwon-Chung, 1999, Chang et al., 1996). However, they did not determine the specific biochemical functions of these genes even though they are necessary for capsule synthesis. Actually, no gene coding for components of the biosynthetic pathways leading to the complete capsule structure has been identified (Doering, 2000). A number of mutants producing thicker or thinner capsule than the wild-type have been described in the literature but no evidence of a structural modification of their capsules have been reported (Jacobson et al., 1982; Jacobson and Tingler, 1994). UDP glucuronate decarboxylase, UDP xylosyltransferase, UDP glucuronyltransferase, UDP glucose dehydrogenase and, more recently α-(1→3) mannosyltransferase activities has been identified in C. neoformans extracts but their involvement in the biosynthesis of the capsule remains to be clearly demonstrated (Doering, 1999; Jacobson, 1987; Jacobson and Payne, 1982, White et al., 1990).

Little is known about the impact of the capsule-polysaccharide structure on the pathophysiology of cryptococcosis, even though several lines of evidence suggest its effect on the virulence of C. neoformans. In vivo in humans, serotypes A vs D, as well as serotype A vs B differ in terms of the type of infection produced (Dromer et al., 1996; Speed and Dunt, 1995). In vitro, various GXM produce various effects in terms of antiphagocytic activity (Small and Mitchell, 1989) or induction of cytokine synthesis by different host cells (Chaka et al., 1997; Lipovsky et al., 1998). Finally, capsule structure seems to affect the in vitro binding of C3 to C. neoformans cells (Washburn et al., 1991; Young and Kozel, 1993). However, how the capsule influences yeast virulence is unknown.

The O-acetylation is one of the most common modifications that occur on sialic acids. They are approximatively 40 members of the sialic acid family that have been identified so far. This diversity is generated by different substituents occuring on this particular 9-carbon carboxylated sugan with an exocyclic chain (Klein et al, O-Acetylation of sialics acids, Biochimie (1988) 80, 49-57).

The relation between sialic acid modifications in developement of maligancy have been studied by Ajit Varki (Mini review, Diversity in the sialic acids, Glycobiology vol.2 no 1, pages 25-40, 1992).

Recently, the methods of purification and analysis have demonstrated that the degree of O-acetylation of sialic acids was variable according to the cellular population. The sialic acids can be O-acetylated in position 4, 7, 8 and/or 9 but the precise functions of such modifications is yet unknown. However, it has been demonstrated that these residues are located outside the cells and they are implicated in cells to cells interactions during the tissular differenciation (i.e. nervous system, kidney, and leucocytes). The presence of the O-acetyl residues are also a protection against the viral and bacterial sialidases.

The use of lectines and antibodies specifically directed against these residues has demonstrated that the O-acetylation of sialic acids is regulated during the developpment and usually modified in tumor cells. By example, a O-acetylated disialoganglioside (GD3) is a biosensor of malignancy cells in humans. Furthermore, the level of O-acetylated sialic acids deregulation in mucines in colic cells during a colorectal cancer or in Hirschsprung disease. A modification of the level of O-acetylated GD3 has been observed in patient suffering of psoriasis or breast cancer. Lastly, although the normal B cells lymphocytes expresses a little quantity of 9-O-acetylated sialic acids, this quantity is increasing in patients suffering of infant lymphoblastic leucemia.

The way of biosynthesis of O-acetylated sialic acid in human is practically unknown. The O-acetyltransferases are difficult to purify and although many attemps, no cDNA encoding for an O-acetyltransferase has been actually identified in human. Only one gene called AT-1 and encoding for a putative carrier of acetyl-CoA and necessary for the biosynthesis of these residus has been recently identified.

### SUMMARY OF THE INVENTION

This invention aids in the fulfilling this need in the art. The inventors have focused on the identification of genes implicated in capsule biosynthesis in order to study the relationship between the C. neoformans capsule structure and virulence.

Herein, the inventors describe the cloning of the first Capsule Synthesis gene, CAS1, coding for an enzyme, whose activity is necessary for the synthesis of a specific sugar epitope on GXM. They demonstrated that its specific deletion influenced GXM structure and demonstrated that CAS1 codes for a new kind of O-acetyltransferase.

More particularly, the inventors has cloned and sequenced two new polypeptides designated hsCAS1 and cnCAS1, which encodes for glycosyltransferase, respectively hsCas1p and cnCas1p.

According to the invention, hsCAS1 designates the human gene, hsCas1p the human polypeptide, both purified and annoted and originating from the human genome and cnCAS1 and cnCas1p respectively the polynucleotide sequence and the polypeptide sequence, both purified and annoted and originating from the Cryptococcus neoformans genome.

In particular, this invention provides a purified polynucleotide comprising the sequence of SEQ ID NO: 1, and also designated as hsCAS1 polynucleotide.

The invention also provides a purified polynucleotide comprising the sequence of SEQ ID NO: 3, and also designated as cnCAS1 polynucleotide.

This invention additionally provides a purified polypeptide comprising an amino acid sequence of SEQ ID NO: 2 and also designated as hsCas1p and a purified polypeptide comprising an amino acid sequence of SEQ ID NO: 4 and also designated as cnCas1p.

The invention includes nucleics acids molecules complementary to these sequences, comprising the complementary RNA, and purified polynucleotides which encodes an allelic variant or a homolog of hsCas1p polypeptide and cnCas1p polypeptide.

The invention includes purified polynucleotide or polynucleotide fragments comprising at least 10 contiguous nucleotides that hybridizes to either strand of a denatured, double-stranded DNA comprising a polynucleotide according to the invention under conditions of high stringency as defined hereinafter.

The invention further includes a polynucleotide derived by in vitro mutagenesis from SEQ ID NO: 1 or SEQ ID NO: 3 and polynucleotide degenerate from SEQ ID NO: 1 or SEQ ID NO: 3 as the result of genetic code. In vitro mutagenesis includes numerous techniques known in the art including, but not limited to, site-directed mutagenesis, random mutagenesis, and in vitro nucleic acid synthesis.

The invention also include a purified polynucleotide sequence comprising the SEQ ID NO:11.

The nucleic acid molecules of the invention, which include DNA and RNA, are referred to herein as “CAS1 nucleic acids” or “CAS1 DNA” or “CAS1 RNA”, and the amino acids encoded by these molecules are referred to herein as “Cas1p polypeptides”. Preferably, the Cas1p polypeptide has an observed nuclear weight of about 108 kDa as determined by electrophoresis).

The invention also provides corresponding nucleic acid molecules, and polypeptides encoded by them, of C. neoformans, which are designated as “cnCAS1 nucleic acids” and “cnCas1p” polypeptides. The invention further includes a polynucleotide derived by in vitro mutagenesis from SEQ ID NO: 3 and polynucleotide degenerate from SEQ ID NO: 3 as the result of genetic code.

In another embodiment, the invention comprises polypeptides encoded by polynucleotide sequences according to the invention or fragments thereof of at least 5 amino acids and defined as above.

In a preferred embodiment of the invention, the invention comprises the family of polypeptides with a glycosyltransferase activity which are encoded by a polynucleotide according to the invention and selected from the group of polynucleotides as above defined.

In still a further embodiment, the invention comprises a polypeptide having a molecular weight of approximately 108 kDA and encoded by a polynucleotide according to the invention. The molecular weight has been determinated experimentaly by the software <<PI/MW tool>> available at the internet web site http://www.expasy.ch/tools/pi_tool.html.

In a preferred embodiment, the invention encompasses a polypeptide having a molecular weight of approximately 108 kDA with an Glycosyltransferase activity encoded by a polynucleotide sequence of SEQ ID NO: 1.

In a preferred embodiment, the invention encompasses a polypeptide having a molecular weight of approximately 108 kDA with an Glycosyltransferase activity encoded by a polynucleotide sequence of SEQ ID NO: 3.

In another preferred embodiment, the invention encompasses a polypeptide having a molecular weight of approximately 108 kDA with an Glycosyltransferase activity with the sequence of SEQ ID NO: 2.

In another preferred embodiment, the invention encompasses a polypeptide having a molecular weight of approximately 108 kDA with an Glycosyltransferase activity with the sequence of SEQ ID NO: 4.

Furthermore, the invention includes a polypeptide according to the invention which is in post translationnally modified form or not.

The polypeptides of the invention can be labeled. Preferably, the labeled polypeptides are in purified form. It is also preferred that the unlabeled or labeled polypeptide is capable of being immunologically recognized by human body fluid containing antibodies.

The polypeptides can be labeled, for example, with an immunoassay label selected from the group of radiactive, enzymatic, fluorescent, chemiluminescent labels, and chromophores.

Additionally, the invention includes polypeptide or peptide fragments having at least 10 amino acids, which are recognized by antibodies directed against a peptide encoded by a polynucleotide sequence corresponding to the polynucleotide sequence of SEQ ID NO: 1 or SEQ ID NO: 3 or fragment thereof.

The invention also encompasses an isolated immunological complex formed between such antibodies and a polypeptide according to the invention. The immunological complex can be labeled with an immunoassay label selected from the group of radiactive, enzymatic, fluorescent, chemiluminescent labels, and chromophores.

The purified antibodies according to the invention which bind to a polypeptide sequence comprising SEQ ID NO: 2 or SEQ ID NO: 4 or fragments, are preferably monoclonal antibodies specifically directed against theses polypeptides but also can be polyclonal, labeled or not.

The invention also includes a recombinant DNA molecule comprising at least one nucleotide sequence defined as SEQ ID NO: 1 and/or SEQ ID NO: 3 under the control of regulatory elements that regulate the expression of of these molecules in a host.

The invention also includes chimeric proteins comprising at least a polypeptide of SEQ ID: 2 and/or SED ID NO: 4.

The invention includes recombinant vector that directd the expression of a nucleic acid molecule comprising a purified ponynucleotide according the invention as above described.

The invention provides a bacteria containing a plasmid pNE8 deposited at the C.N.C.M. on May 10, 2001 under the accession number I-2666.

The invention also provides a plasmid pNE95 deposited at the C.N.C.M. on May 10, 2001 under the accession number I-2667.

The present invention includes a cell host such as a bacteria containing SEQ ID NO:1 or SEQ ID NO:3 or a recombinant DNA molecule comprising SEQ ID NO:1 or SEQ ID NO:3 under the control of regulatory elements that regulates the expression of the glycosyltransferase in said host.

The invention provides a cell host transfected or transduced byrecombinant vector that directs the expression of nucleic acid molecule selected from the group consisting of the purified polynucleotide according to the invention and as above defined and the host cell transfect or transduced by such recombinant vector.

The invention also contemplates probes and primers, such as at least 8 nucleotides of SEQ ID NO: 1 or SEQ ID NO: 3.

In a preferred embodiment, the invention encompasses the following primers:

(SEQ ID NO: 5)

| | |
|---|---|
| Cas1b: | 5′-TATCTCTTCCTCGCCGACAG-, |

(SEQ ID NO: 6)

| | |
|---|---|
| Cas1e: | 5′-TAGCCATTCAGTGATTTCGC-, |

(SEQ ID NO: 7)

| | |
|---|---|
| Cas1m: | 5′-TGTATTTGGAGCCGATAGCCATAGTCGC-, |

(SEQ ID NO: 8)

| | |
|---|---|
| Cas11: | 5′-TATGGGCTATCTCTTCCTCGCCGACA-, |

(SEQ ID NO: 9)

| | |
|---|---|
| HuCASR: | 5′-CCCCTTGGTCACTGTATGGTTCATGG-, and |

(SEQ ID NO: 10)

| | |
|---|---|
| HuCASR2: | 5′-CCATGAACCATACAGTGACCAAGGGG-. |

Furthermore, the invention includes a composition comprising at least one polynucleotide sequence designated as SEQ ID NO: 1 and/or SEQ ID NO: 3, and coding for a glycosyltransferase or fragment thereof or at least any polynucleotides as above mentioned.

Still further, the invention encompasses a composition comprising at least one polypeptide encoded by a polynucleotide sequence of SEQ ID NO: 2 and/or SEQ ID NO: 4. fragment thereof.

In another embodiment, the invention comprises a method for the production of Cas1p polypeptides comprising culturing a host cell according to the invention under conditions promoting expression, and recovering the polypeptide from the host cell or the culture medium.

In a preferred embodiment, the host cell is selected from the group consisting of bacterial cells, parasite cells, fungal cells and eukariotic cells.

The invention contemplates a method of production of CAS1p polypeptide comprising culturing a host cell according to the invention under conditions to promote expression and recovering polypeptide from the host cell or the culture medium.

In still a further embodiment, the invention comprises a diagnostic method for the predictive prognosis of cancer in a patient comprising the following steps

a) Preparing two tissue samples, the first tissue sample being derived from an organ suspected to be tumorous and a second tissue sample being derived from a healthy patient;

b) Optionnally making the genomic RNA or DNA contained in the cells of the tissue sample of step a) available to hybridization;

c) Amplifying the genomic RNA or DNA with at least one primer according to the invention,

d) Measuring the level of transcription of the DNA sequence corresponding to the hsCAS1 gene contained in genomic DNA of said tissue samples,

e) Detecting the deregulation of transcription that have occured in the biosynthesis of o-Acetyltransferase in the sample derived from the patient suspected to be tumorous by comparing the resulting product of step d) derived respectively from the first and the second tissue sample.

An example of cancer which can be detected by this method is the colorectal cancer.

This invention also provides an in vitro diagnostic method for the detection of the presence or absence of antibodies, which bind to an antigen comprising the hsCas1p polypeptides of the invention or mixtures of the polypeptides. The method comprises contacting the antigen with a biological fluid for a time and under conditions sufficient for the antigen and antibodies in the biological fluid to form an antigen-antibody complex, and then detecting the formation of the complex. The detecting step can further comprise measuring the formation of the antigen-antibody complex. The formation of the antigen-antibody complex is preferably measured by immunoassay based on Western blot technique, ELISA (enzyme linked immunosorbent assay), indirect immunofluorescent assay, or immunoprecipitation assay.

The invention also provides a diagnostic kit for the detection of the presence or absence of antibodies, which bind to the hsCas1p polypeptide of the invention or mixtures thereof, and means for detecting the formation of immune complex between the antigen and the antibodies. The antigens and the means are present in an amount sufficient to perform the detection.

The invention provide a therapeutic method for targeting in vitro a molecule of interset with an anti-tumoral activity in mammal cells comprising measuring the level of transcription of hsCAS1 in cells where the level of transcription of hsCAS1 is deregulated and testing the anti tumoral activity of the molecule of interest.

The polypeptides of htis invention are thus useful as a portion of a diagostic composition for detecting the presence of antibodies to antigenic proteins associated with C.neoformans.

In addition, the hsCas1p polypeptides can be used to raise antibodies for detecting the presence of antigenic proteins associated with C. neoformans.

The polypeptides of the invention can also employed to raise neutralizing antibodies that eitheir inactivate C. neoformans, reduce the viability of the C. neoformans in vivo, or inhibit or prevent C. neoformans replication. The ability to elicit C. neoformans neutralizing antibodies is especially important when the proteins and polypeptides of the inventions are used in immunising or vaccinating compositions to activate the B-cell arm of the immune response or induce a cytotoxic T lymphocyte response (CTL) in the recipient host.

The invention provides a method for detecting the presence or absence of C. neoformans comprising:

a) Contacting a sample suspected of containing genetic material of C. neoformans with at least one nucleotide probe according to the invention, and

b) Detecting hybridization between said nucleotidic probe and the genetic material in the sample

Wherein said probe is complementary to the full length sequence of the purified CAS1 nucleic acid of the invention.

In another embodiment, the invention comprises a diagnostic method for the predictive prognosis of colorectal cancer in a patient comprising the following steps:

a) Preparing two tissue samples from a patient, the first tissue sample being derived from an organ different than the colon and a second tissue sample being derived from the colon of said patient;

b) Optionnally making the genomic RNA or DNA contained in the cells of the tissue sample of step a) available to hybridization;

c) Amplifying the genomic RNA or DNA with at least one primer according to the invention,

d) Measuring the level of transcription of the DNA sequence corresponding to the hsCAS1 gene contained in genomic DNA of said tissue samples,

e) Detecting the deregulation of transcription that have occured in the biosynthesis of o-Acetyltransferase in the sample derived from the colon by comparing the resulting product of step d) derived respectively from the first and the second tissue sample.

The measure of the level of transcription of the DNA sequence according to the invention is, in a preferred embodiment, done by the technique of quantitative RT-PCR.

According to the invention, a deregulation of the level of transcription of the O-acetylation of sialic acids during cancerogenesis is a sign of the presence of tumor cells. Under a normal fold, the patient cell which have been preleved can be considered as a tumor cell.

The deregulation can be compared with a normal level which can be found by measuring the level of transcription of hsCAS1 gene in healthy cells.

The sequence according to the invention can therefore be used for a early diagnosis and prognosis of cancer, in particular, the colorectal cancer.

The industrial application of the sequences according to the invention could be by example the use of hsCAS1 as a biomarker of the carcinogenesis in the humans cells.

In a preferred embodiment, the hsCAS1 sequence could be use to measure the level of expression of this gene during the carcinogenesis by quantitative RT-PCR.

The DNA sequence specific for this gene can also be spotted in arrays together with other gene potentially involved in the carcinogenesis. These arrays could be probed with labeled RNA isolated from putative tumors cells.

In a preferred embodiment, the invention encompasses DNA chips with at least a sequence according to the invention for the detection of tumor cells and the use of the polynucleotide sequences according to the invention for the use and the make of DNA chips.

In another embodiments, an antibody specific of the hsCas1p could be also used to measure the expression of the gene in normal and tumor cells.

The invention also comprises a method for identifying an active agent of interest that interacts with the activity of the polypeptide according to the invention comprising the following steps:

a) Contacting a biological sample containing mammal cells with a polypeptide according to the invention in the presence or absence of a candidate agent; and

b) Comparing the binding of the said polypeptide in absence of said agent,

c) Optionally, testing the activity of said selected agent on a preparation of a cellular extract comprising sialic acids.

The invention comprises a test of screening of an inhibitor molecule inhibiting the activity of a glycosyltransferase according to the invention comprising the following steps:

a) Contacting a biological sample containing mammal cells with a polypeptide according to the invention in the presence or absence of a inhibitor molecule of interest; and

b) Comparing the binding of the said polypeptide in absence of said molecule.

It is to be understood that both the foregoing general description and the following detailed description are exemplary and explanatory only and are not restrictive of the invention, as claimed.

### BREF DESCRIPTION OF THE DRAWINGS

FIG. 1. Comparison of the strains NE28 (MATαcas1-Δura5) and JEC43 (MATαCAS1 ura5) by light microscopy (A and B); immunofluorescence labeling with the monoclonal antibody CRND-8 (C and D); and India ink negative-staining of the capsule (E and F).

FIG. 2. is a alignment of the amino acids sequences of the human (hsCas1p) and the C. neoformans (cnCas1p) orthologues indicating identical (*), strongly similar (:) or weakly similar (.) residues.

FIG. 3. contains hydropathy profiles of cnCas1p, hsCas1p and dmCas1p determined by TMHMM1.0 analysis.

FIG. 4. Depicts the disruption of CAS1. (A) Physical map of the CAS1 region and strategy used to construct the disruption cassette. (B) Southern-blot analysis of the DNA digested by HindIII and hybridized with the CAS1-specific probe. Lane A: JEC43; Lane B to H: independent cas1-Δ strains. (C) Immunoblot analysis of the various mutants and constructs using the different GXM-specific antibodies indicated. The plasmid pNE10 differs from pCnTEL-1 in that it lacks the NotI fragment containing the telomeric sequences.

FIG. 5. shows ¹H NMR spectra of GXM isolated from JEC21 (a), NE30+pNE10 (b) and NE30+CAS1 (c). The spectra were recorded at 600.13 MHz.

FIG. 6. shows 2D [¹H, ¹³C] HSQC spectra of GXM isolated from JEC21 (a), NE30+pNE10 (b) and NE30+CAS1 (c) recorded at 600.13 MHz. The lower spectra represent the anomeric regions of the mannosyl residues. HSQC cross-peaks for the O-acetyl groups are not shown. The complete signal assignment is only labeled in spectrum (b). Only labels relevant for O-acetylation are shown in (a) and (c). * Represents signals from a second unassigned GXM of approximately 15% of the main GXM.

FIG. 7. Depicts a virulence assay. (A) Survival of mice infected with 10⁷ viable yeast cells from representative strains (B) Survival of mice infected with 10⁶ viable yeast cells from representative strains (▪) MATαCAS1; (▴) MATa CAS1; (□) MATαcas1-Δ; (?)MATa cas1-Δ.

FIG. 8. Shows the polynucleotide sequence of human gene hsCAS1 also designated SEQ ID NO: 1.

FIG. 9. Shows the polypeptide sequence of human protein hsCas1p also designated SEQ ID NO:2.

FIG. 10. Shows the polynucleotide sequence of Cryptococcus neoformans gene cnCAS1 also designated SEQ ID NO:3.

FIG. 11. Shows the polypeptide sequence of Cryptococcus neoformans protein cnCas1p also designated SEQ ID NO:4.

FIG. 12: Nucleotidic sequence hsCAS1b around the homolog of hsCAS1 located on chromosom 9 of the human genome also designated SEQ ID NO:11.

### DETAILED DESCRIPTION OF THE INVENTION

It has been determined that the capsule is certainly the most obvious virulence factor for Cryptococcus neoformans. The main capsule constituents are glucuronoxylomannans (GXM). Although few studies have focused on GXM chemistry, very little is known about their genetics. Using a monoclonal antibody specific to a sugar epitope, the inventors isolated a capsule-structure mutant strain and cloned by complementation a gene named CAS1 that codes for a membrane protein and necessary for the biosynthesis of the O-acetyl residues. The mutants strains of C. neoformans with no enzymatic activity associated with CAS1 synthetizes a capsule completely de-O-acetylated. Although no sequence homology was found with any known protein in the different databases, protein analysis using the PROPSEARCH software classified Cas1p as a glycosyltransferase. Cas1p is a very well evolutionary conserved protein as the inventors identified one orthologue in the human genome, one in the drosophila melanogaster genome and four in the plant genome of Arabidopsis thaliana. There is no sequence similary to CAS1 in the S.cerevisiae genome. Analysis of the capsule structure after CAS1 deletion showed that it is required for GXM O-acetylation. The inventors hypothesized that CAS1 code for a new kind of O-acetyltransferase.

Furthermore, the inventors has isolated in C.neoformans genome a polynucleotide designated CAS8 homologues to AT-1 human gene which demonstrated that the biosynthesis of O-acetyl residues are conserved both between C.neoformans and human.

This invention provides for the first time a complete human polynucleotide sequence designated as hsCAS1 located on chromosome 7 of human genome encoding for an O-acetyltransferase and polynucleotide fragment thereof, the polypeptide hsCas1p encoded by the gene, polypeptides derived from the protein, and their use in diagnostic. The sequence of hsCAS1 has been determined to have 51% homology with the sequence of CAS1 of C. neoformans.

The level of expression of hsCAS1 in tumoral tissues or pre-tumoral tissues can be a marker of the evolution of cancerogenesis. It can be used as a diagnostic tool and/or prognostic tool for the determination of the evolution of the tumor in tissue in the patient.

The level of expression of hsCAS1 can be measured by example by RT-PCR quantitative. The nucleotide sequence of the complementary DNA of hsCAS1 can be used for the determination of two probes useful for RT-PCR studies. In a preferred embodiment theses probes could be huCASR4 and huCASNco1.

The purified oligonucleotides of the invention are useful as primers for use in amplification reactions or as nucleic acid probes

In a specific embodiment of the invention, the purified polynucleotides are useful for the detection and the prognostic of several cancers by measuring the level of expression and/or transcription of the polynucleotide according to the invention in several tissues.

As used herein, the term “polynucleotides” according refers to a genus of polypeptides that further encompasses proteins having the amino acid sequence of SEQ ID NO: 1 and/or SEQ ID NO: 3, as well as those proteins and polypeptides having a high degree of similarity (at least 90% homology) with such amino acid sequences and which proteins and polypeptides are immunoreactive and the complementary sequence and/or the sequences of polynucleotides, which hybridize to the referred sequences in high stringent conditions and which are used for detecting cancer in human.

Thus, the polynucleotides of SEQ ID NO: 1 and SED ID NO:3 and their fragments can be used to select nucleotide primers notably for an amplification reaction, such as the amplification reactions further described.

In addition, hsCas1p polypeptides refers to the gene products of the nucleotides of SEQ ID NO: 2 and SEQ ID NP: 4.

The terms <<isolated>> and <<purified>> according to the invention refer to a level of purity that is achievable using current technology. The molecules of the invention do not need to be absolutely pure (i.e., contain absolutely no molecules of other cellular macromolecules), but should be sufficiently pure so that one of ordinary skill in the art would recognize that they are no longer present in the envirronment in which they were originally found (i.e., the cellular middle). Thus, a purified or isolated molecule according to the present invention is one that have been removed from at least one other macromolecule present in the natural environment in which it was found. More preferably, the molecules of the invention are essentially purified and/or isolated, which means that the composition in which they are present is almost completely, or even absolutely, free of other macromolecules found in the environment in which the molecules of the invention are originally found. Isolation and purification thus does not occur by addition or removal of salts, solvents, or elements of the periodic table, but must include the removal of at least some macromolecules.

A hsCas1p polypeptide “variant” as referred to herein means a polypeptide subbstantially homologous to native hsCas1p polypeptides, but which has an amino acid sequence different from that of native hsCas1p polypeptides because of on or more deletions, insertions, or substitutions.

The variant amino acid sequence preferably is at least 80% identical to a native hsCas1p polypeptide amino acod sequence, most preferably at least 90% identical.

The percent identity can be determined, for example by comparing sequence information using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482, 1981). The preferred default parameters for the GAP program include: (1) a unary comparison matrix (containing a value of 1 for identities and 0 for non-identities) for nucleotides, and the weighted comparison matrix of Gribskov and Burgess (Nucl. Acids Re. 14:6745, 1986) as described by Schwartz and Dayhoff, eds., (Atlas of Protein Sequence and Structure, National Biomedical Search Fundation, pp. 353-358, 1979) (2) a penalty of 3.0 for each gap and an additional 0.10 penalty for each symbol in each gap; and (3) no penalty for end gaps.

Variants can comprise conservatively substitued sequences, meaning that a given amino acid residue is replaced by a residue having similar physiochemical characteristics. Examples of conservative substitutions include substitution of one aliphatic residue for another, such as Ile, Val, Leu, Ala for one another, or substitutions of one polar residue for another, such as between Lys and Arg, Glu and Asp, or Gln and Asn. Other such conservative substitutions, for example, substitutions of entire regions having similar hydrophobicity characteristics, are well known. Naturally occuring hsCas1p polypeptide variants are also encompassed by the invention. Examples of such variants are proteins that result from alternate mRNA splicing events or from proteolytic cleavage of the hsCas1p polypeptides. Variations attributable to proteolysis include, for example, differences in the termini upon expression in different types of host cells due to proteolytic removal of one or more terminal aminoacids from the hsCas1p polypeptides. Variations attributable to frameshifting include, for example, differences in the termini upon, expression in different types of host cells dus to different amino acids of hsCas1p polypeptides.

Due to the known degeneracy of the genetic code, wherein more than one codon can encode the same amino acid, a DNA sequence can vary from that shown in SEQ ID NO:2 and still encode a hsCAS1p polypeptide having the amino acid sequence of SEQ ID NO: 1. Such variant DNA sequences can result from silent mutations (e.g., occurring during PCR amplification), or can be the product of deliberate mutagenesis of a native sequence.

The invention thus provides equivalent isolated DNA sequences, encoding hsCas1p polypeptides, selected from: (a) DNA derived from the coding region of a native hsCas1 gene; (b) cDNA comprising the nucleotide sequence of SEQ ID NO: 1; (c) DNA capable of hybridization to a DNA of (a) under conditions of moderate stringency and which encode hsCAS1p polypeptides; and (d) DNA which is degenerate as a result of the genetic code to a DNA defined in (a), (b) or (c) and which encodes hsCas1p polypeptides. hsCas1p polypeptides encoded by such DNA equivalent sequences are encompassed by the invention.

DNA that is equivalent to the DNA sequence of SEQ ID NO:1 will hybridize under moderately stringent conditions to the double-stranded native DNA sequence that encode polypeptides comprising amino acid sequences of SEQ ID NO:2. Examples of hsCas1p polypeptides encoded by such DNA, include, but are not limited to, hsCas1p polypeptide fragments and hsCas1p polypeptides comprising inactivated N-glycosylation site(s), inactivated protease processing site(s), or conservative amino acid substitution(s), as described above. hsCas1p polypeptides encoded by DNA derived from other species, wherein the DNA will hybridize to the complement of the DNA of SEQ ID NO: 1 are also encompassed by this invention.

Recombinant expression vectors containing a nucleic acid sequence encoding hsCas1p polypeptides can be prepared using well known methods. The expression vectors include a hsCas1 DNA sequence operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences, which control transcription and translation initiation and termination. Nucleotide sequences are “operably linked” when the regulatory sequence functionally relates to the hsCas1 DNA sequence. Thus, a promoter nucleotide sequence is operably linked to a hsCas1 DNA sequence if the promoter nucleotide sequence controls the transcription of the hsCas1 DNA sequence. The ability to replicate in the desired host cells, usually conferred by an origin of replication, and a selection gene by which transformants are identified can additionally be incorporated into the expression vector.

In addition, sequences encoding appropriate signal peptides that are not naturally associated with hsCas1p polypeptides can be incorporated into expression vectors. For example, a DNA sequence for a signal peptide (secretory leader) can be fused in-frame to the hsCas1 nucleotide sequence so that the hsCas1p polypeptide is initially translated as a fusion protein comprising the signal peptide. A signal peptide that is functional in the intended host cells enhances extracellular secretion of the hsCas1p polypeptide. The signal peptide can be cleaved from the hsCAS1p polypeptide upon secretion of hsCAS1p polypeptide from the cell.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids. Commercially available vectors include those that are specifically designed for the expression of proteins. These include pMAL-p2 and pMAL-c2 vectors, which are used for the expression of proteins fused to maltose binding protein (New England Biolabs, Beverly, Mass., USA).

Promoter sequences commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980; and EP-A-36776), and tac promoter (Maniatis, Molecular Cloning: A Laboratory Manual, Cold Spring Harbor Laboratory, p. 412, 1982).

Suitable host cells for expression of hsCas1p polypeptides include prokaryotes, yeast or higher eukaryotic cells. Appropriate cloning and expression vectors for use with bacterial, fungal, yeast, and mammalian cellular hosts are described, for example, in Pouwels et al. Cloning Vectors: A Laboratory Manual, Elsevier, New York, (1985). Cell-free translation systems could also be employed to produce hsCas1p polypeptides using RNAs derived from DNA constructs disclosed herein.

It will be understood that the present invention is intended to encompass the previously described proteins in isolated or purified form, whether obtained using the techniques described herein or other methods. In a preferred embodiment of this invention, the hsCas1p polypeptides are substantially free of human tissue and human tissue components, nucleic acids, extraneous proteins and lipids, and adventitious microorganisms, such as bacteria and viruses. It will also be understood that the invention encompasses equivalent proteins having substantially the same biological and immunogenic properties. Thus, this invention is intended to cover serotypic variants of the proteins of the invention.

Depending on the use to be made of the hsCas1p polypeptides of the invention, it may be desirable to label them. Examples of suitable labels are radioactive labels, enzymatic labels, fluorescent labels, chemiluminescent labels, and chromophores. The methods for labeling proteins and glycoproteins of the invention do not differ in essence from those widely used for labeling immunoglobulin. The need to label may be avoided by using labeled antibody to the antigen of the invention or anti-immunoglobulin to the antibodies to the antigen as an indirect marker.

Once the hsCas1p polypeptides of the invention have been obtained, they can be used to produce polyclonal and monoclonal antibodies reactive therewith. Thus, a protein or polypeptide of the invention can be used to immunize an animal host by techniques known in the art. Such techniques usually involve inoculation, but they may involve other modes of administration. A sufficient amount of the protein or the polypeptide is administered to create an immunogenic response in the animal host. Any host that produces antibodies to the antigen of the invention can be used. Once the animal has been immunized and sufficient time has passed for it to begin producing antibodies to the antigen, polyclonal antibodies can be recovered. The general method comprises removing blood from the animal and separating the serum from the blood. The serum, which contains antibodies to the antigen, can be used as an antiserum to the antigen. Alternatively, the antibodies can be recovered from the serum. Affinity purification is a preferred technique for recovering purified polyclonal antibodies to the antigen from the serum.

Monoclonal antibodies to the antigens of the invention can also be prepared. One method for producing monoclonal antibodies reactive with the antigens comprises immunizing a host with the antigen; recovering antibody producing cells from the spleen of the host; fusing the antibody producing cells with myeloma cells deficient in the enzyme hypoxanthine-guanine phosphoribosyl transferase to form hybridomas; selecting at least one of the hybridomas by growth in a medium comprising hypoxanthine, aminopterin, and thymidine; identifying at least one of the hybridomas that produces an antibody to the antigen; culturing the identified hybridoma to produce antibody in a recoverable quantity; and recovering the antibodies produced by the cultured hybridoma.

These polyclonal or monoclonal antibodies can be used in a variety of applications. Among these is the neutralization of corresponding proteins. They can also be used to detect viral antigens in biological preparations or in purifying corresponding proteins, glycoproteins, or mixtures thereof, for example when used in a affinity chromatographic columns.

The hsCAS1p polypeptides can be used as antigens to identify antibodies to C. Neoformans in materials and to determine the concentration of the antibodies in those materials. Thus, the antigens can be used for qualitative or quantitative determination of the virus in a material. Such materials of course include human tissue and human cells, as well as biological fluids, such as human body fluids, including human sera. When used as a reagent in an immunoassay for determining the presence or concentration of the antibodies to C. Neoformans, the antigens of the present invention provide an assay that is convenient, rapid, sensitive, and specific.

More particularly, the antigens of the invention can be employed for the detection of C. Neoformans by means of immunoassays that are well known for use in detecting or quantifying humoral components in fluids. Thus, antigen-antibody interactions can be directly observed or determined by secondary reactions, such as precipitation or agglutination. In addition, immunoelectro-phoresis techniques can also be employed. For example, the classic combination of electrophoresis in agar followed by reaction with anti-serum can be utilized, as well as two-dimensional electrophoresis, rocket electrophoresis, and immunolabeling of polyacrylamide gel patterns (Western Blot or immunoblot). Other immunoassays in which the antigens of the present invention can be employed include, but are not limited to, radioimmunoassay, competitive immunoprecipitation assay, enzyme immunoassay, and immunofluorescence assay. It will be understood that turbidimetric, calorimetric, and nephelometric techniques can be employed. An immunoassay based on Western Blot technique is preferred.

Immunoassays can be carried out by immobilizing one of the immunoreagents, either an antigen of the invention or an antibody of the invention to the antigen, on a carrier surface while retaining immunoreactivity of the reagent. The reciprocal immunoreagent can be unlabeled or labeled in such a manner that immunoreactivity is also retained. These techniques are especially suitable for use in enzyme immunoassays, such as enzyme linked immunosorbent assay (ELISA) and competitive inhibition enzyme immunoassay (CIEIA).

When either the antigen of the invention or antibody to the antigen is attached to a solid support, the support is usually a glass or plastic material. Plastic materials molded in the form of plates, tubes, beads, or disks are preferred. Examples of suitable plastic materials are polystyrene and polyvinyl chloride. If the immunoreagent does not readily bind to the solid support, a carrier material can be interposed between the reagent and the support. Examples of suitable carrier materials are proteins, such as bovine serum albumin, or chemical reagents, such as gluteraldehyde or urea. Coating of the solid phase can be carried out using conventional techniques.

To further achieve the objects and in accordance with the purposes of the present invention, a kit capable of diagnosing an C. Neoformans infection is described. This kit, in one embodiment, contains the DNA sequences of this invention, which are capable of hybridizing to viral RNA or analogous DNA sequences to indicate the presence of an C. Neoformans pathogen or infection. Different diagnostic techniques can be used, which include, but are not limited to: (1) Southern blot procedures to identify cellular DNA, which may or may not be digested with restriction enzymes; (2) Northern blot techniques to identify RNA extracted from cells; and (3) dot blot techniques, i.e., direct filtration of the sample through an ad hoc membrane, such as nitrocellulose or nylon, without previous separation on agarose gel. Suitable material for dot blot technique can be obtained from body fluids including, but not limited to, serum and plasma, supernatants from culture cells, or cytoplasmic extracts obtained after cell lysis and removal of membranes and nuclei of the cells by centrifugation.

The following plasmids were deposited at the Collection Nationale de Cultures de Microorganismes (C.N.C.M.), of Institut Pasteur, 28, rue du Docteur Roux, F-75724 Paris, Cedex 15, France, on May 10, 2001, and assigned the following Accession Nos.:

| PLASMID | ACCESSION NO. |
|---|---|
| pNE8 | I-2666 |
| pNE95 | I-2667 |

The oligonucleotides according to the present invention hybridize specifically with a DNA or RNA molecule comprising all of the part of one polynucleotide among SEQ ID NO:1 or SEQ ID NO:3 under stringent conditions.

As an illustrative embodiment, the stringent hybridization conditions used in order to specifically detect a polynucleotide according to the present invention are advantageously the following:

Prehybridization and hybridization are performed at 68° C. in a mixture containing:

5×SSPE (1×SPE is 0.18 M NaCl, 10 mM NaH₂PO₄.);

5× Denhardt's solution;

0.5% (w/v) sodium dodecyl sulfate (SDS); and

100 μg ml-1 salmon sperm DNA.

The washings are performed as follows:

a) Two washing at laboratory temperature for 10 min in the presence of 2×SSPE and 0.1% SDS;

b) One washing at 68° C. for 15 min. in the presence of 1×SSPE and 0.1% SDS; and

c) One washing at 68° C. for 15 min. in the presence of 0.1×SSPE and 0.1% SDS.

A example of low stringency conditions for hybridizations in the present invention are washings in steps b) and c) performed at 50° C.

The moderate stringency conditions in the present inventions had an identical protocol as the conditions for high stringent hybridization but steps b) and c) are performed at 55° C. or 60° C.

The non-labeled polynucleotides or oligonucleotides of the invention can be directly used as probes.

Nevertheless, the polynucleotides or oligonucleotides are generally labeled with a radioactive element (³²P, ³⁵S, ³H, ¹²⁵I) or by a non-isotopic molecule (for example, biotin, acetylaminofluorene, digoxigenin, 5-bromo-desoxyuridin, fluorescin) in order to generate probes that are useful for numerous applications. Examples of non-radioactive labeling of nucleic acid fragments are described in the French Patent No. FR 78 10975 or by Urdea et al. or Sanchez-Pescador et al. 1988.

Other labeling techniques can also be found in French Patents 2 422 956 and 2 518 755. The hybridization step may be performed in different ways (Mattews et al., 1988). A general method comprises immobilizing the nucleic acid that has been extracted from the biological sample on a substrate (nitrocellulose, nylon, and polystyrene) and then incubating, in defined conditions, the target nucleic acid with the probe. Subsequent to the hybridization step, the excess amount of the specific probe is discarded, and the hybrid molecule formed are detected by an appropriate method (radioactivity, fluorescence, or enzyme activity measurement) as well known by the man of the art.

The oligonucleotide fragments according to the invention can be prepared by cleavage of the polynucleotides of SEQ ID NO:1 or SEQ ID NO:3 by restriction enzymes, as described in Sambrook et al. in 1989.

Another technique for the isolation of the nucleic acids of the invention containing at most 20 nucleotides (or 200 bp if these molecule are double-stranded) comprises the following steps:

Synthesizing DNA using the automated method of β-cyanoethylphosphoramidite;

a) Cloning the thus obtained nucleic acids in an appropriate vector; and

b) Purifying the nucleic acid by hybridizing to an appropriate probe according to the present invention.

A chemical method for producing the nucleic acids according to the present invention, which have a length of more 200 nucleotides (or 200 bp if these molecule are double-stranded) comprises the following steps:

a) Assembling the chemically synthesized oligonucleotides having different restriction sites at each end;

b) Cloning the thus obtained nucleic acids in an appropriate vector; and

c) Purifying the nucleic acid by hybridizing to an appropriate probe according to the present invention.

The oligonucleotides according to the present invention can also be used as antigens to obtain an immune response in a mammal if they contain some particular epitopes of interest. Therefore, the oligonucleotides of the present invention can be useful as immunogenic nucleotides included in a composition with an acceptable carrier.

The oligonucleotides according to the present invention can also be used in a detection device comprising a matrix library of probes immobilized on a substrate, the sequence of each probe of a given length being localized in a shift of one or several bases, one from the other, each probe of the matrix library thus being complementary to a distinct sequence of the target nucleic acid. Optionally, the substrate of the matrix can be a material able to act as an electron donor, the detection of the matrix positions in which hybridization has occurred being subsequently determined by an electronic device. Such matrix library of probes and method of specific detection of a target nucleic acid are described in the European patent application No. 0 713 016, or PCT Application WO 95 33846, or also PCT Application WO 95 11995 (Affymax Technologies), and in PCT Application WO 97 02357 (Affymetrix Inc.), and also in U.S. Pat. No. 5,202,231 (Drmanac), said patents and patent application being herein incorporated by reference.

The present invention also pertains to a family of recombinant plasmid such pNE8, pNE 25, pNE 28 or pPNE95 containing at least a nucleic acid according to the invention.

In a preferred embodiment, the invention comprises the plasmid pNE8 deposited at the C.N.C.M. on May 10, 2001 under the accession number I-2666.

In another preferred embodiment, the invention comprises the plasmid pPNE95 deposited at the C.N.C.M. on May 10, 2001 under the accession number I-2667.

The polypeptides according to the invention can also be prepared by conventional method of chemical synthesis, either in a homogenous solution or a solid phase. As an illustrative embodiment of such chemical polypeptide synthesis techniques the homogenous solution technique described by Houbenweyl et al. in 1974 may be cited.

The polypeptide according to the invention can be characterized by binding onto an immunoaffinity chromatography column on which polyclonal or monoclonal antibodies directed to the polypeptide or fragments have been previously immobilized.

Another object of the present invention comprises a polypeptide produced by genetic engineering techniques or a polypeptide synthesized chemically as above described.

The monoclonal antibodies according to the invention can be prepared from hybridomas according to the technique described in Kohler and Milstein in 1975.

The polyclonal antibodies can be prepared by immunization of a mammal, especially a mouse or a rabbit, with a polypeptide or a fragment of interest of a polypeptide according to the invention that is combined with an adjuvant, and then by purifying specific antibodies contained in the serum of the immunized animal on a affinity chromatography column on which has previously been immobilized the polypeptides or the fragments of interest that has been used has an antigen.

Example of a method for the amplification of the oligonucleotides according to the invention, The SDA amplification technique:

The Strand Displacement Amplification (SDA) technique (Walker et al, 1992) is an isothermal amplification technique based on the ability of a restriction enzyme to cleave one of the strands at its recognition site (which is under a hemiphosphorothioate form) and on the property of a DNA polymerase to initiate the synthesis of a new strand from the 3′OH end and generated by the restriction enzyme and on the property of this DNA polymerase to displace the previously synthesized strand being localized downstream. The SDA method comprises two mains steps: (a) the synthesis in the presence of dCTP-alpha-S, of DNA molecules that are flanked by the restriction sites that may be cleaved by an appropriate enzyme; (b) the exponential amplification of these DNA molecules modified as such by enzymes cleavage, strand displacement and copying of the displacement strands. The steps of cleavage, strand displacement and copying are repeated a sufficient number of times in order to obtain an accurate sensitivity of the assay.

The SDA technique was initially realized using the restriction endonuclease HincII but is now generally practiced with an endonucleasefrom Bacillus stearothermophillus (BSOBI) and a fragment of a DNA polymerase which is devoid of and 5′ 3′ exonuclease activity isolated from Bacillus cladotenax (exo-Bca) [=exo-minus-Bca]. Both enzymes are able to operate at 60° C. and the system is now optimized in order to allow the use of dUTP and the decontamination by UDG. When using this technique as described by (Spargo et al.). In 1996, the doubling time of the target DNA is of 26 seconds and the amplification rate is 10¹⁰ after an incubation of 156 min at 60° C.

The SDA amplification technique is easier to perform than PCR (a single thermostated water bath device is necessary) and is faster than the other amplification methods. An illustrative example can be found in Walker G. T. et al., (1992, Nucleic Acids Res., 1691-1696.) and (Spargo C. A. et al., 1996, Mol. And Cell. Probes, 10 :247-256.).

Another method for the measurement of the level of transcription of a gene according to the invention is disclosed in Sambrook et al. (Sambrook J. et Russel D. W., 2001, molecular cloning. A laboratory manual. Third edition. Protocol 8., amplification of cDNA generated by reversed transcription of mRNA, pages 8.46-8.53 and Protocol 15., quantitative PCR, pages 8.86-8.95. Cold Sring Harbor Laboratory press).

The analyze of the alignment of the amino acids sequences of the human (hsCas1p) and the C. neoformans (cnCas1p) paralogues of hsCas1p were searched by tblastn analysis at the NCBI (http://www.ncbi.nlm.nih.gov).

Two convergently oriented identical homologous sequences were identified on the human chromosom 9. The putative hsCAS1bp protein shared up to 40% of identity with hsCas1p.

Materials and Methods

Experimental Procedure

Strains and Culture Media

The C. neoformans strains used in this study are listed in Table I.

TABLE I
C. neoformans strains used in this study.

| Strains | Genotype | Source or ref. |
|---|---|---|
| JEC21 | MATα | (Moore and Edman, 1993) |
| JEC20 | MATa | (Moore and Edman, 1993) |
| JEC156 | MATa ade2 ura5 | (Wickes and Edman, 1995) |
| JEC43 | MATα ura5 | (Wickes and Edman, 1995) |
| B-4500 | MATα | (Kwon-Chung et al., 1992) |
| NE23 | MATα cas1-1 | This study |
| NE28 | MATα cas1-1 ura5 | This study |
| H99 | MATα | J. Heitman |
| TYCC33 | MATα cap59-Δ::ADE2 ura5 | (Chang and Kwon-Chung, |
| | | 1994) |
| TYCC77 | MATα cap64-Δ::ADE2 ura5 | (Chang et al., 1996) |
| TYCC122 | MATα cap60-Δ::ADE2 ura5 | (Chang and Kwon-Chung, |
| | | 1998) |
| TYCC150 | MATα cap10-Δ::ADE2 ura5 | (Chang and Kwon-Chung, |
| | | 1999) |
| NE30 | MATa cas1-Δ::ADE2 ura5 | This study |
| NE31 | MATa cas1-Δ::ADE2 ura5 | This study |
| JEC33 | MATα lys2 | (Wickes and Edman, 1995) |
| JEC50 | MATa ade2 | (Wickes and Edman, 1995) |
| JEC52 | MATα lys2 ura5 | (Wickes and Edman, 1995) |

The strains were routinely cultured on YPD medium at 30° C. (Sherman, 1992) Minimum medium (YNB) contained 6.7 g of yeast nitrogen base without amino acids (Difco, Detroit, Mich.) and 20 g of glucose per liter of water. Induction medium contained 1.7 g of yeast nitrogen base without amino acids and without ammonium sulfate (Difco), 1.5 g of asparagine and 20 g of glucose per liter of buffer (12 mM in NaHCO₃ 35 mM in MOPS (pH 7.1) (Granger et al., 1985). 5-Fluoroorotic acid (5-FOA) medium contained 6.7 g of nitrogen base (Difco), 1 g of 5-FOA, 50 mg of uracil, and 20 g of glucose per liter. Bacterial strains Escherichia coli DH5α (Hanahan, 1983) and XL1-blue (Stratagene, La Jolla, Calif.) were used for the propagation of all plasmids.

Monoclonal Antibodies

The anticapsular monoclonal antibodies E1 (Dromer et al., 1987), CRND-8 kindly provided by T. Shinoda (Tokyo, Japan) (Ikeda et al., 1996), 4H3, 2H1 and 5E4 kindly provided by A. Casadevall (Casadevall and Scharff, 1991) (Albert Einstein College of Medicine, Bronx, N.Y.) were used in immunoblotting and immunofluorescence experiments.

DNA and RNA Handling

Genomic DNA purification was carried out as previously described (Garcia-Hermoso et al., 1999). The kit systems RNeasy® Mini and Oligotex™ kits respectively, (Qiagen S. A., Courtaboeuf, France) were used to purify total RNA and mRNA from C. neoformans cells. Reverse transcriptase-polymerase chain reaction (RT-PCR) experiments were performed with the Access RT-PCR System (Promega, Madison, Wis.) using total RNA as the substrate and the primers Cas1b (5′-TATCTCTTCCTCGCCGACAG) (SEQ ID NO:5) and Cas1e (5′-TAGCCATTCAGTGATTTCGC) (SEQ ID NO:6). The primers Act1L (5′-CCTTGGTCATTGACAATGGC) (SEQ ID NO:12) and Act1R (5′-GATCGATACGGAGGATAGCG) (SEQ ID NO: 13) were used to amplify a part of the actin gene cDNA as an internal control for each experiment. Southern blotting and colony hybridization was carried out using standard protocol (Sambrook et al., 1989). Probe labeling, hybridization, washing, and detection of hybridized bands or colonies were performed using the DIG non-radioactive nucleic acid labeling and detection system (Roche Diagnostic, Meylan, France) according to the manufacturer's instruction. DNA was sequenced by Cybergene (Evry, France) using synthetic primers. Programs of the University of Wisconsin Genetics Computer Group (Madison, Wis.) were used for analysis of nucleic acid sequences (Devereux et al., 1984). Protein transmembrane domains were predicted using the program TMHMM1.0 (Sonnhammer et al., 1998). PROPSEARCH software (Hobohm and Sander, 1995) was used to identify protein families. Restriction endonuclease digestion and ligation were carried out using standard methods, as recommended by the suppliers.

3′ and 5′-RACE Analysis

The C and N termini of Cas1p were determined using the Smart RACE system kit from Clontech (Palo Alto, Calif.). The first-strand cDNA synthesis and PCR amplification of the cDNA fragments were performed according to the manufacturer's instructions using 1 μg of mRNA from C. neoformans JEC21 grown in YPD liquid medium as the starting material. The primers Cas1m (5′-TGTATTTGGAGCCGATAGCCATAGTCGC) (SEQ ID NO:7) and Cas1l (5′-TATGGGCTATCTCTTCCTCGCCGACA) (SEQ ID NO:8) were used for the 5′ and 3′-end amplifications respectively. The cDNA fragments were then cloned into the pGEM®-T Easy Vector (Promega, Madison, Wis.) and sequenced. The 3′ and 5′ ends of human homologue cDNA was cloned using the Marathon-Ready™ cDNA (Clontech) and huCASR (5′-CCCCTTGGTCACTGTATGGTTCATGG) (SEQ ID NO: 9) and huCASR2 (5′-CCATGAACCATACAGTGACCAAGGGG) (SEQ ID NO:10) primers. The PCR program was: 94° C. for 1 min., 5 cycles of 30 s at 94° C., 4 min. at 72° C., 5 cycles of 30 s at 94° C., 4 min. at 70° C., 25 cycles of 20 s at 94° C., 4 min. at 68° C. The resulting PCR RACE products were ligated into the pGEM®-T Easy Vector and sequenced.

Polysaccharide Studies

Cells were grown in liquid induction medium for 3 days at 30° C., then pelted by centrifugation and capsule polysaccharides were precipitated by adding 3 volumes of 95% ethanol to the culture supernatant. GXM extracts were finally prepared as previously described (Cherniak et al., 1991). Before NMR analysis, GXM (15-25 mg) were sonicated for 10 min., exchanged twice in 99.5% deuterium oxide (D₂O) and lyophilized. The samples were finally dissolved in 0.5 ml of 99.96% D₂O (Sigma, St. Louis, Mo.).

Nuclear Magnetic Resonance (NMR) Spectroscopy

NMR spectra were recorded at 57° C. with a Bruker Avance 600 NMR spectrometer using a 5-mm [¹H, ¹³C] inverse detection probe equipped with pulsed field gradients and operated at 600.13 and 150.913 MHz, respectively. ¹H NMR spectra were acquired with a spectral width of 3000 Hz, time domain 32 K, 128 acquisitions, repetition time of 6.5 s. 2D NMR spectra were acquired using standard Bruker pulse sequences (xwinnmr 2.6 program). Double quantum filtered [¹H, ¹H] COSY, TOCSY, ROESY and NOESY spectra were obtained in the phase-sensitive mode. The spectral width was 3000 Hz, 256 increments of 16 acquisitions were acquired, each containing 2K data points. The acquisition time was 340 ms and the relaxation delay 1.5 s. Squared sine-bell functions shifted by π/2 were applied for data processing in t₁ and t₂. Zero filling was used to expand the data matrix to 1K in the t₁ dimension. The mixing time for the TOCSY spectra was either 40 or 150 ms and for ROESY spectra 70 ms. [¹H, ¹³C] one-bond shift correlation spectra were obtained in the ¹H detection mode using a gradient enhanced HSQC pulse sequence. Acquisition parameters were: spectral width in t₁ 12000 Hz and in t₂ 3000 Hz, optimization for ¹J_{C,H} coupling constants of 140 Hz, acquisition of 512 increments in t₁ each consisting of 64 acquisitions of 2K data points, acquisition time 170 ms, relaxation delay 1s. ¹³C decoupling during acquisition were achieved by GARP-1. A sine-bell function shifted by π/2 were applied in t₂ and a Gaussian-Lorentzian function in t₁. Zero filling to 1 K was used in t₁ prior to Fourier transformation. Cross-peak volumes were determined for relative comparison of cross-peaks in the HSQC spectra. [¹H, ¹³C] multiple bond heteronuclear correlation spectra (HMBC) were acquired similarly to the HSQC spectra. No ¹³C decoupling were applied. The spectral width in t₁ was 15000 Hz. Experiments were optimized for ¹J_{C,H} long-range coupling constants of approximately 2 Hz. The number of acquisitions per increment was 128.

Mutagenesis and Screening for Cas Mutant Strains

C. neoformans strain B-4500 was removed from −70° C. and transferred onto a YPD plate as independent colonies. After 2 days of incubation, cells from one colony were resuspended in water. Appropriate dilutions were then plated on YPD and immediately irradiated with an UV lamp (254 nm) to obtain 90% survival. After 3 days of incubation in the dark, each colony was picked up and cultured individually in 100 μl of induction medium in 96-well plates. On each plate, 4 wells were inoculated with one colony of the strain B-4500 as control. After 2 days, 100 μl of a 40% glycerol solution were added to each culture and mixed. The 96-well plates were then stored at −20° C. until tested. Each culture was tested by ELISA as follows. Using a multichannel pipette, 20 μl of each culture were transferred to a 96-well MultiScreen filtration system plate (Millipore, Bedford, Mass.) (Dromer et al., 1993). The cells were washed twice with phosphate-buffered saline (PBS) (Sambrook et al., 1989) and then incubated with the anticapsule monoclonal antibody, CRND-8 (Ikeda et al., 1996) and an alkaline phosphatase-conjugated goat anti-mouse immunoglobulin M (IgM) secondary antibody (Sigma) for 30 min. After two washes with PBS, the cells were transferred to a new 96-well plate and incubated with p-nitrophenyl phosphate as recommended by the supplier (Sigma). The optical density was recorded using an ELISA plate reader (Labsystem multiscan plus, Uppsala, Sweden). The presence of a capsule was checked by Indian ink negative staining. Finally, the putative Cas mutants were checked for CRND-8 reactivity by immunofluorescence assay using strain B-4500 as the positive control. The CRND-8 negative mutant strain isolated by this method, named NE23, was then crossed on V-8 juice agar with the strain JEC156 using a classical procedure (Kwon-Chung et al., 1982). After selection on 5-FOA medium, an Ura⁻ derivative strain, named NE28, was isolated.

Cloning of CAS1 from C. neoformans var. neoformans

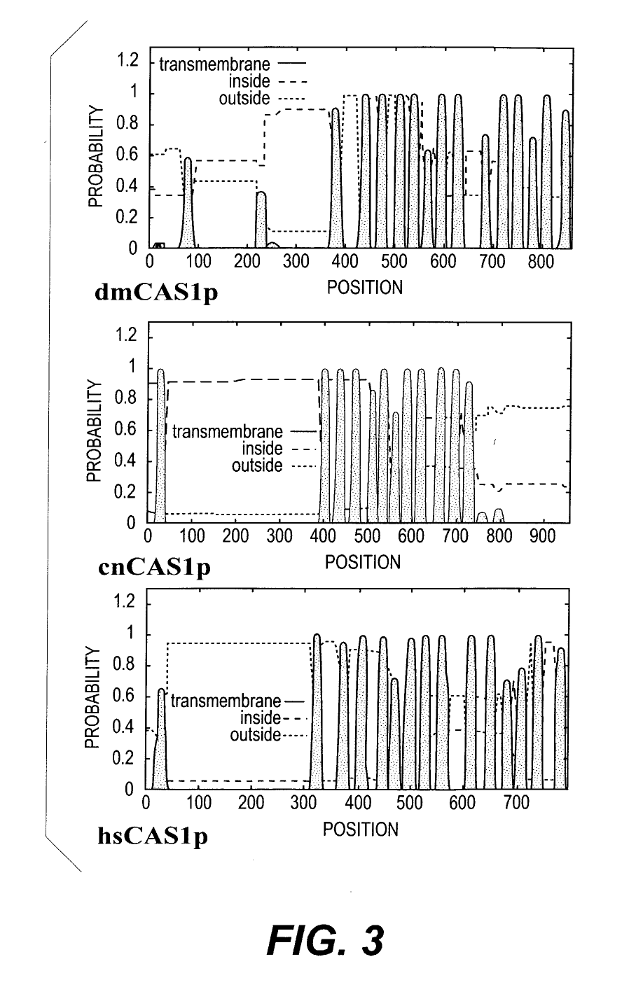

The strain NE28 was transformed by electroporation (Edman and Kwon-Chung, 1992) with a genomic library constructed in the plasmid pCnTEL-1 and kindly provided by B. Wickes (San Antonio, Tex.). The NE28 transformants were grown on induction medium plates for 2 days and then transferred onto nitrocellulose paper (Sartorius, Göttingen, Germany). The blots were dried using a hair dryer for 1 min. and incubated for 2 h in a 50 mM Tris (pH 7.5), 200 mM NaCl, 0.1% Tween 20 and 5% nonfat dry milk buffer (TBS-Tween-milk buffer). After two washes with TBS-Tween, the blots were incubated with the CRND-8 antibody and horseradish peroxidase-conjugated goat anti-mouse IgM secondary antibody (Sigma) for 1 h. The membranes were then washed twice with TBS-Tween and once with TBS. CRND-8 positive clones were detected using the chemioluminescent substrate ECL (Amersham Pharmacia Biotech, Buckinghamshire, UK) as recommended by the manufacturer. DNA from each of the CRND-8 positive NE28 transformants was extracted from cells grown on minimum medium, digested with NotI, ligated and used to transform E. coli (Chang and Kwon-Chung, 1994). The plasmids, purified from the resulting E. coli transformants, were subsequently used to transform C. neoformans NE28. Finally, among a total of 56,000 Ura⁺ transformants that were screened, 7 had the following properties: their cells reacted with antibody CRND-8 as determined by immunoblotting; they were plasmid dependent, in that the cells from their Ura⁻ derivatives no longer reacted with CRND-8 antibody; and they produced CRND-8 positive transformants when the corresponding plasmid was introduced into C. neoformans strain NE28. The restriction maps of the 7 plasmids indicated that the inserted DNA contained common sequences. A physical map of a portion of the 5.5-kb insert from plasmid pNE1 is shown in FIG. 3. Gene disruption

The CAS1 gene was disrupted using the positive-negative selection protocol described by Chang and Kwon-Chung (Chang and Kwon-Chung, 1994). First, the BamHI/BamHI fragment from pNE1 was subcloned into pUC18 resulting in pPNE8. The plasmid pNE25 was then constructed by subcloning the EagI/SmaI fragment from pPNE8 into pUC 18. The EcoRV/StuI fragment was then replaced by the SmaI/SmaI fragment from the plasmid pRCD28, kindly provided by J. Heitman (Durham, N.C.), which carries the ADE2 marker (Sudarshan et al., 1999), to result in pNE27. Finally, pNE28 was constructed by subcloning the pNE27 XbaI/EcoRI fragment containing the disruption cassette at the XbaI/EcoRI sites of pCIP3, kindly provided by K. J. Kwon-Chung (Bethesda, Md.) which contains the URA5 marker (Chang and Kwon-Chung, 1994). After electroporation of strain JEC156, the strains that had integrated the disruption cassette in the correct position were screened by PCR using the primers Cas1h (5′-GCAGTTCAAACCAAGGAGG-3′) (SEQ ID NO: 17) and Ade2a (5′-GACAAGTACATCGAGAGGCT-3′) (SEQ ID NO:18) and checked by Southern blotting using the CAS1 gene as the probe.

Immunofluorescence and Immunoblotting Experiments

Immunofluorescence assays were carried out as previously described (38). For immunoblotting experiments, 3 μl of a 10⁷ cells/ml suspension of a 2-days minimum medium culture, were spotted on a nitrocellulose membrane and dried using a hair dryer for 1 min. The membranes were then treated as described above.

Pulse-Field Gel Electrophoresis (PFGE)

C. neoformans chromosome plugs for PFGE were prepared as previously described (Dromer et al., 1993) and the chromosomes were separated on a 0.8% Agarose gel (Tris acetate EDTA IX) using a CHEF DR II apparatus (Biorad, Hercules, Calif.) with the following parameters: 14° C., 96 h, pulse from 400 to 800 s, 2 V/cm.

Capsule Thickness Measurement

Successive rounds of crossing using the strains JEC50 (MATa ade2), JEC52 (MATa lys2 ura5), JEC33 (MATa lys2), JEC156 (MATa ade2 ura5) were performed starting with the cas1-Δ strain NE30. Out of the 28 Ura⁺, Ade⁺, Lys⁺ randomly chosen strains finally selected, 7 were MATα CAS1, 8 were MATα cas1-Δ, 7 were MATa CAS1 and 6 were MATa cas1-Δ. To determine the thickness of the capsules, cells were grown at 30° C. overnight in induction medium and washed twice in PBS. The mean value of the distance from the cell wall to the outer border of the capsule for each strain was calculated from 20 cells in a suspension of India ink under 100× magnification using a grid with a resolution of 0.12 μm (Rivera et al., 1998).

Virulence Study

After each cross, 4 strains representative for each genotype (i.e. MATαcas1-Δura5 CAS1, MATαcas1-Δura5 cas1-Δ, MATa CAS1 and MATa cas1-Δ) were randomly chosen. Six-week-old male BALB/c mice (Charles River, St.-Aubin-lès-Elbeuf, France) were injected in the lateral tail vein with cells from each of the yeast strains (7/group) as previously described (Lortholary et al., 1999a). Mortality was monitored daily.

Results

### EXAMPLE 1

Cloning of CAS1

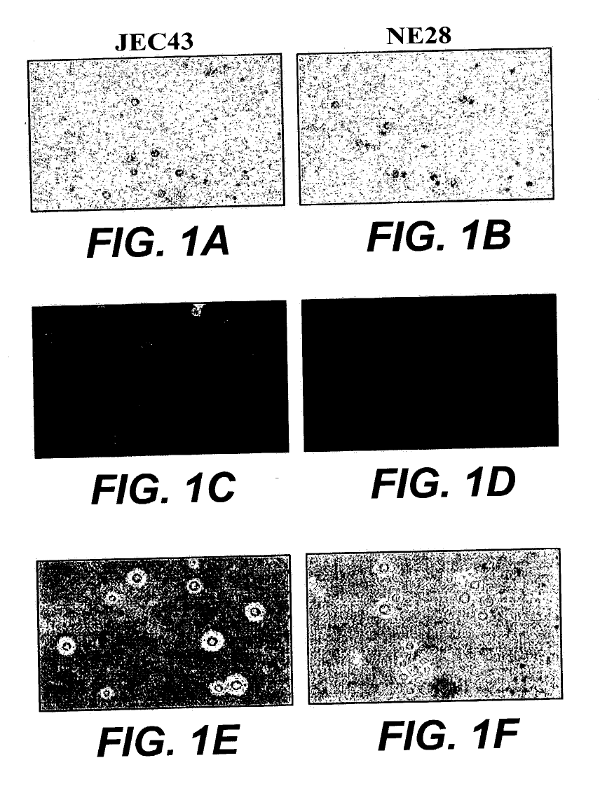

The isolation and description of acapsular, hypocapsular and hypercapsular mutants were previously reported (9,13,14). We isolated NE28 (Table I) a mutant strain, which has a modified capsule structure strain NE28 that did not react with the GXM-specific monoclonal antibody CRND-8 (FIG. 1).

The hsCAS1 gene was isolated by complementing the strain NE28's inability to react with monoclonal antibody CRND-8. The 7 CRND-8-positive transformants yielded plasmids with inserts 5.5 to 8 KB long. Restriction maps of the cloned fragments, subcloning, and complementation experiments established that the gene of interest was located on a 4.5-kb BamHI/BamHI fragment. Sequencing of the whole fragment revealed two large open reading frames (ORF) of 758 and 1064 BP, which could correspond to two large exons of the gene. Using two primers each specific to one of these ORF, we amplified, cloned and sequenced the complete corresponding cDNA fragment (see Materials and Methods). This sequence contained a 2880-bp ORF of which encoded a putative 108-kDa protein. Alignment of this sequence with the genomic DNA sequence revealed the presence of 8 short introns. The polyadenylation site was located 71 nucleotides downstream from the stop codon. By sequencing 5 independent clones after 5′ RACE cDNA amplification (see Materials and Methods), it was determined that the 5′ end of the transcript was located between −44 and -60 upstream from the ATG.

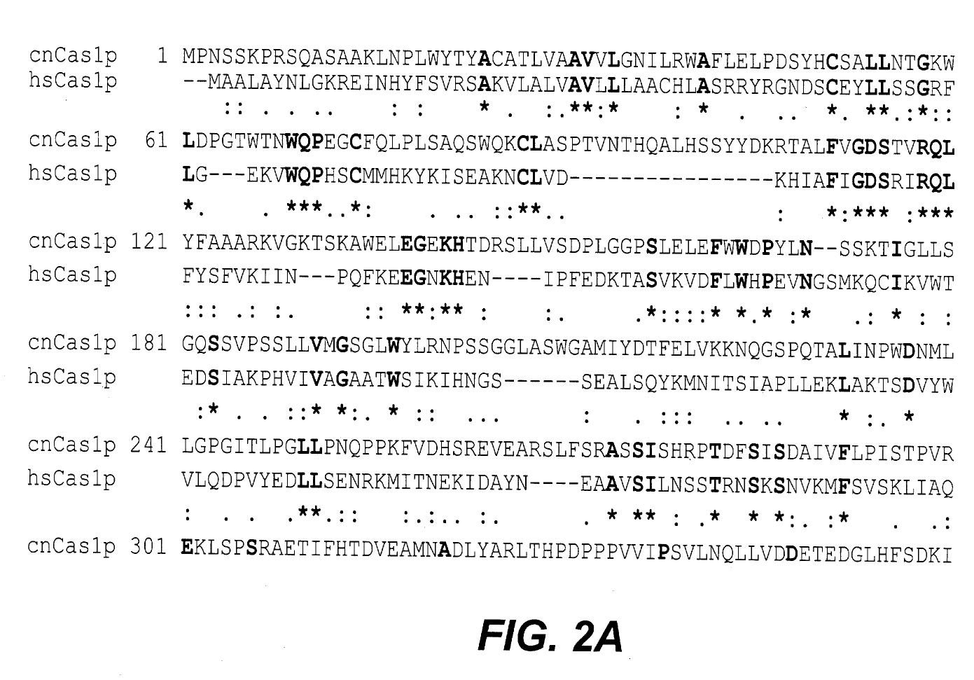

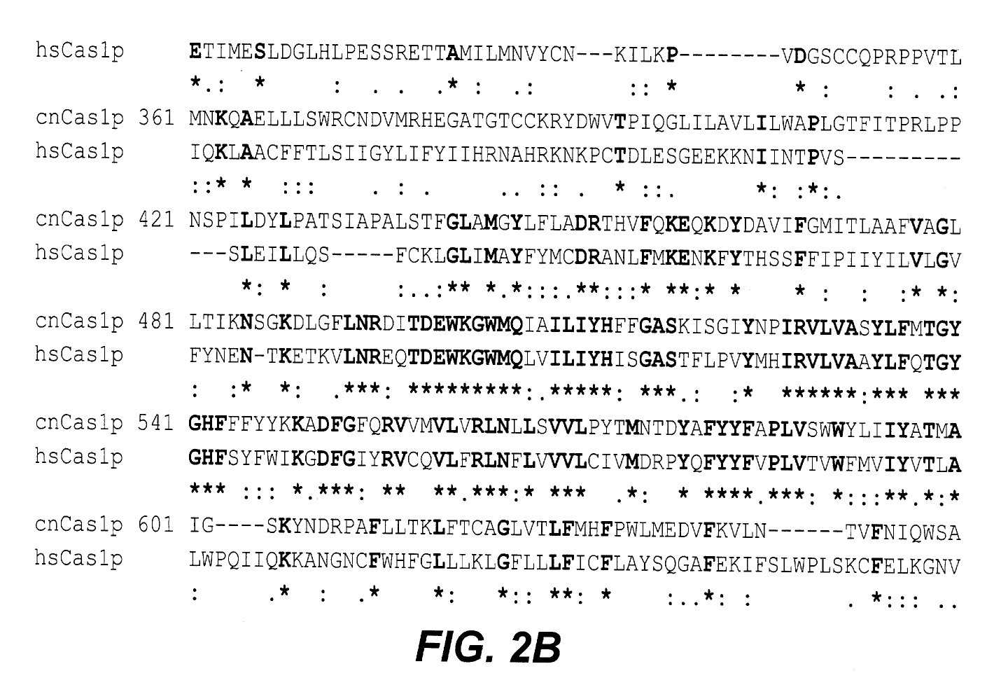

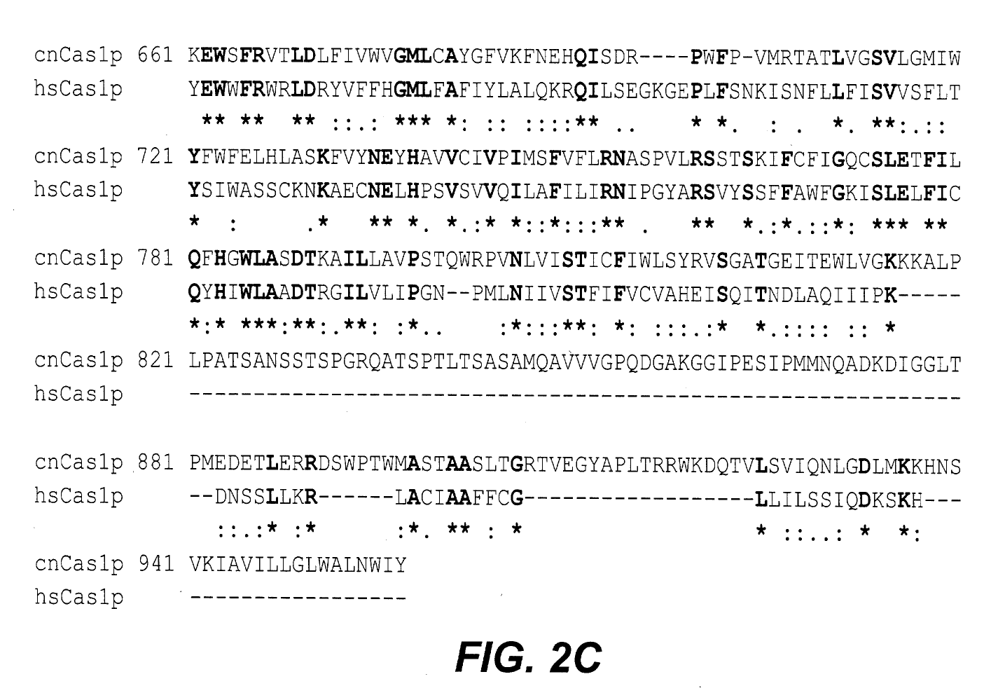

Comparison of the sequence with the available databases revealed no homology with any known protein. However, we identified one similar sequence in the human genome located on the chromosome 7. The complete corresponding cDNA from a human placenta cDNA library was amplified by PCR and the complete sequence of the human orthologue (hsCAS1) was determined. The amino-acid sequence was 22% identical and 51% similar to that of the C. neoformans protein sequence (see alignment of the sequences on FIG. 2). The Drosophila melanogaster genome also contained one orthologue gene (locus CG2938) located on chromosome X (19% identical and 46% similar). We also identified four orthologue genes on the Arabidopis thaliana genome on chromosomes I, II, III and V. The partial sequences of these proteins were very similar to each other (90% similarity) and shared an average 70% similarity with the C. neoformans protein sequence.

The analysis of the cnCas1p amino-acid sequence revealed the presence of two putative N-glycosylation sites in positions 3-6 and 169-172. Moreover, the sequence AARKVGKT (SEQ ID NO:11) in position 124-131 matched the ATP/GTP-binding site motif A (P-loop) consensus sequence [AG]-x(4)-G-K-[ST] characteristic of some ATP- or GTP-binding proteins (45) that can be present in some proteins binding other nucleotides. However, this sequence was not conserved in the different Cas1p orthologues.

According to the program TMHMM1.0 (Sonnhammer et al., 1998), Cas1p is a membrane protein with 12 putative transmembrane domains. As shown in FIG. 3, the human and fly orthologues also resembled membrane proteins with 15 putative transmembrane domains. The positions of these domains are very well conserved, with an isolated N-terminal domain and 11 central domains. In cnCas1p, however, the C-terminal is predicted to be cytoplasmic whereas the C-terminal of the human and fly orthologues contained 3 more transmembrane spannings.

The inventors used the PROPSEARCH software to analyze the Cas1p sequence. Among the twelve best hits obtained, eight were glycosyltransferases (Table II).

TABLE II
Protein identified belonging to the same family as Cas1p by PropSearch analysis.

| Protein | Organism | Dist | Function | Reference |
|---|---|---|---|---|
| MdoH | E. coli | 8.23 | Glycosyltransferase | (Loubens et al., 1993) |
| YhjO | E. coli | 8.25 | Glycosyltransferase | (Sofia et al., 1994) |
| Pmt2 | S. cerevisiae | 8.71 | Glycosyltransferase | (Lussier et al., 1995) |
| Env | BIV | 9.43 | Coat polyprotein | (Garvey et al., 1990) |
| HrpM | P. syringae | 9.52 | Glycosyltransferase | (Mukhopadhyay et al., 1988) |
| Env | BIV | 9.68 | Coat polyprotein | (Garvey et al., 1990) |
| Tscc | H. sapiens | 10.04 | Na—Cl cotransporter | (Simon et al., 1996) |
| Isp4 | S. pombe | 10.15 | Peptide transporter | (Sato et al., 1994) |
| Pmt3 | S. cerevisiae | 10.17 | Glycosyltransferase | (Immervoll et al., 1995) |
| Pmt1 | S. cerevisiae | 10.35 | Glycosyltransferase | (Strahl-Bolsinger et al., 1993) |
| BcsA | A. xylinum | 10.38 | Glycosyltransferase | (Standal et al., 1994) |
| AcsA | A. xylinum | 10.43 | Glycosyltransferase | (Wong et al., 1990) |

Thus, this analysis indicated that Cas1p is of the same family as Pmt mannosyltransferases responsible for the first step of Saccharomyces cerevisiae mannoprotein O-glycosylation (Review in (Strahl-Bolsinger et al., 1999)).

### EXAMPLE 2

CAS1 of the Different Serotypes

Southern-blotting experiments using CAS1 as the probe revealed that a homologue of this gene was also present in the genomes of serotypes A, B and C (data not shown). We cloned the serotype A CAS1 homologue by colony hybridization of a PstI partial genomic library of the strain H99. The sequence of this gene was very similar to its serotype D counterpart (94% identity and 99% similarity at the amino-acid level). Moreover, the serotype A CAS1 gene was put under the control of the serotype D promoter and the construct was used to transform strain NE28. The resulting colonies were all CRND-8-positive, showing that the serotype A CAS1 gene could complement the phenotype of the serotype D mutant. The same probe was used to identify the location of CAS1 in C. neofomans serotype D and A genomes after PFGE separation of the chromosomes of strains JEC21, JEC20 and H99. The probe hybridized to only one of the two smallest chromosomes of the strains JEC21 and JEC20, and the smallest chromosome of strain H99 (data not shown). Thus, most probably, only one copy of this gene was present in the genome. No close homologue could be revealed by Southern-blot analysis with either high or low stringency conditions. Moreover, a BLAST search against the partial genome sequence from the “Cryptococcus neoformans Genome Project” at the Stanford Genome Technology Center (http://sequence-www.stanford.edu/group/C.neoformans/index.html) did not revealed any CAS1 homologous sequence other than CAS1 itself.

### EXAMPLE 3

Regulation of CAS1 Transcription

Previous studies have shown that at least 4 genes are necessary for GXM biosynthesis. Thus, when CAP10, CAP59, CAP60 or CAP64 are deleted, the resulting strains have an acapsular phenotype (Chang and Kwon-Chung, 1994; Chang and Kwon-Chung, 1998; Chang and Kwon-Chung, 1999; Chang et al., 1996). Using RT-PCR, we showed that the deletion of any one these genes had no influence on the level of CAS1 transcription as compared to the original strain (data not shown). Moreover, the level of CAS1 transcription was not changed when the cells were grown in a minimum medium or capsule-induction medium (data not shown).

### EXAMPLE 4

Disruption of CAS1

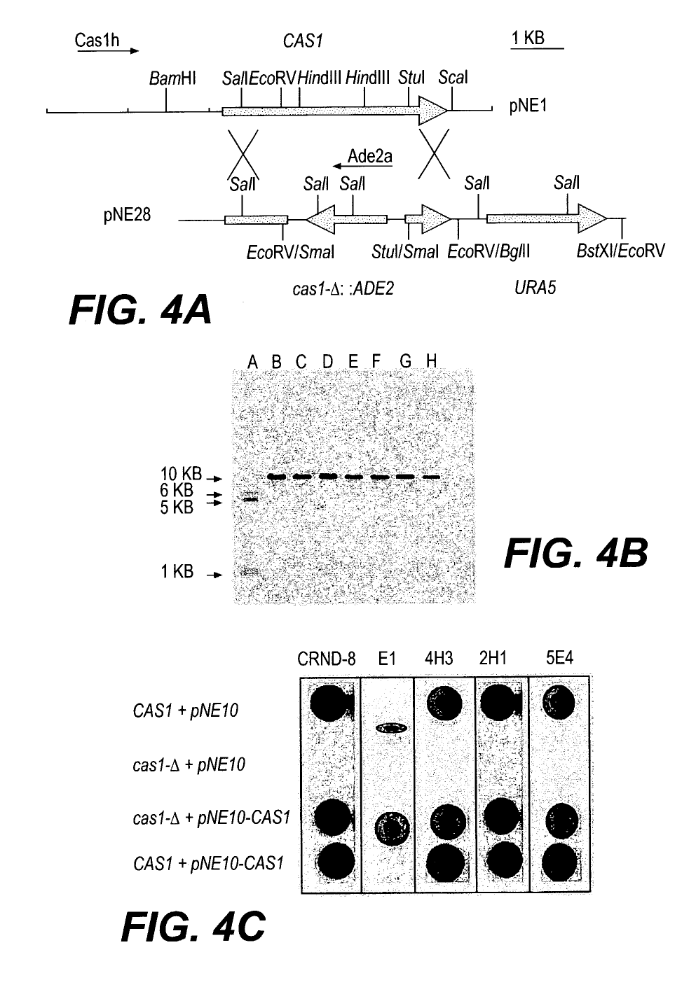

The plasmid pNE28 containing the disruption cassette (see FIG. 4A) was used to transform the strain JEC156. The transformants were selected on 5-FOA medium screening for the Ade⁺, Ura⁻ phenotype. After 3 weeks of incubation at room temperature, two kinds of transformants were visible. The majority of them were slightly pink, indicating episomal replication of the plasmid, and about 10% of them were white, indicating integration of the plasmid. A total of 56 white transformants were tested for their reactivity with the CRND-8 antibody; 8 negative clones were found. PCR analysis showed that the cassette was correctly integrated into the genome and Southern blotting showed that there was only one copy in each. We finally obtained 7 independent disrupted strains (see FIG. 4B). As with the strain NE28, various cas1-Δ strains did not react with any of the five anti-capsular monoclonal antibodies tested (see FIG. 4C). When CAS1 was reintroduced into the cells via an episomal plasmid, the transformant cells reacted with monoclonal antibody E1 while the original strain was completely negative. We tested 3 different independent cas1-□ strains and obtained the same results. When the original strain JEC43 was transformed with the plasmid bearing CAS1, the phenotype of the transformant was unchanged even with E1. The same result was obtained with the strains NE30 and JEC43 and the serotype A CAS1 gene (data not shown).

### EXAMPLE 5

GXM Structure Analysis

TABLE III
¹H and ¹³C chemical shifts of glycolytic residues in the GXM isolated from
the original (JEC21), reconstructed (NE30 + CAS1) and mutant strains
(NE30 + pNE10).

| Strain | | | | | |
|---|---|---|---|---|---|
| position | GlcA | Xyl | Man | Man^{Xyl} | Man^{GlcA} |
| JEC21 | | | | | |
| H-1/C-1 | 4.47/104.3 | 4.36/105.6 | 5.17/104.3 | 5.21/103.7 | 4.23/102.7 |
| H-2/C-2 | 3.39/75.0 | 3.32/75.4 | 4.22/72.6 | 4.23/80.7 | 4.27/80.3 |
| H-3/C-3 | 3.47/78.0 | 3.42/78.2 | 4.03/80.4 | 4.09/78.9 | 4.07/79.5 |
| H-4/C-4 | 3.60/73.6 | 3.63/72.0 | 3.85/69.0 | 3.69/69.9 | 3.79/69.1 |
| H-5/C-5 | 3.65/79.6 | 4.00 (eq), 3.26 (ax)/67.5 | 4.05/73.4 | 3.85/76.4 | 4.13/72.7 |

| | | | |
|---|---|---|---|
| H-6/C-6 | — | — | 3.80-3.88/63.5 (de-O-acetylated) |
| | | | 4.30-4.35/66.5 (O-acetylated) |
| 6-O-ac | — | — | 2.18, 2.20/22.3 |

NE30 + CAS1

| | | | | | |
|---|---|---|---|---|---|
| H-1/C-1 | 4.46/104.4 | 4.37/105.8 | 5.15/104.1 | 5.19/103.7 | 5.22/102.7 |
| H-2/C-2 | 3.36/75.2 | 3.29/75.2 | 4.22/72.7 | 4.20/81.0 | 4.25/80.5 |
| H-3/C-3 | 3.45/78.1 | 3.39/78.1 | 4.01/80.5 | 4.08/79.0 | 4.06/79.8 |
| H-4/C-4 | 3.57/73.0 | 3.62/71.8 | 3.84/68.9 | 3.68/69.7 | 3.82/68.9 |
| H-5/C-5 | 3.64/79.9 | 3.99 (eq), 3.26 (ax)/67.5 | 3.96/75.9^{a} | 3.83/76.2 | 4.02/75.7^{a} |
| | | | 4.06/73.2^{a} | | 4.11/73.2^{a} |

| | | | |
|---|---|---|---|
| H-6/C-6 | — | — | 3.80-3.89/63.3 (de-O-acetylated) |
| | | | 4.32-4.38/66.4 (O-acetylated) |
| 6-O-ac | — | — | 2.20/22.2 |

NE30 + pNE10

| | | | | | |
|---|---|---|---|---|---|
| H-1/C-1 | 4.48/104.3 | 4.38/105.7 | 5.19/104.5 | 5.17/103.8 | 5.23/102.6 |
| H-2/C-2 | 3.39/75.1 | 3.32/75.3 | 4.24/72.3 | 4.23/80.8 | 4.27/79.9 |
| H-3/C-3 | 3.46/78.1 | 3.44/78.2 | 3.97/81.1 | 4.08/78.7 | 4.10/79.9 |
| H-4/C-4 | 3.59/74.2 | 3.65/71.8 | 3.83/68.8 | 3.68/69.8 | 3.82/68.9 |
| H-5/C-5 | 3.65/79.3 | 4.00 (eq), 3.29 (ax)/67.8 | 3.96/76.0 | 3.83/76.1 | 4.02/75.9 |

| | | | |
|---|---|---|---|
| H-6/C-6 | — | — | 3.78-3.88/63.2, 63.5 |

^{a} Two resonances for Man and Man^{GlcA} H-5/C-5 were detected due to incomplete 6-O-acetylation: The first and the last rows for each strain refer to the 6-de-O-acetylated and to the 6-O-acetylated mannosyl residues, respectively. The majority of Man residues were 6-O-acetylated and the majority of the Man^{GlcA} residues were 6-de-O-acetylated.

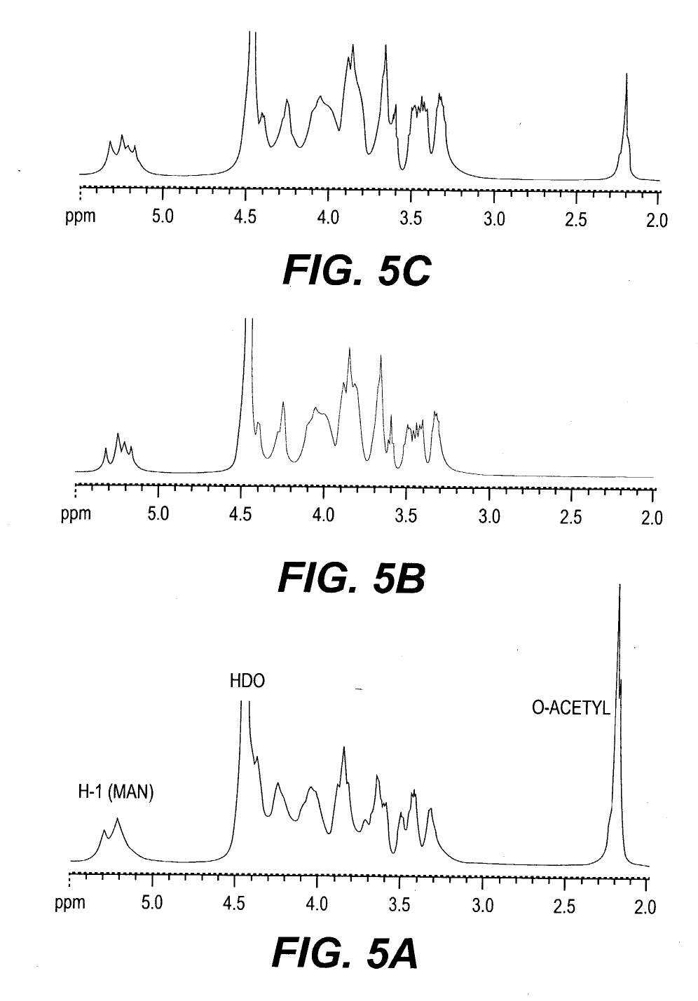

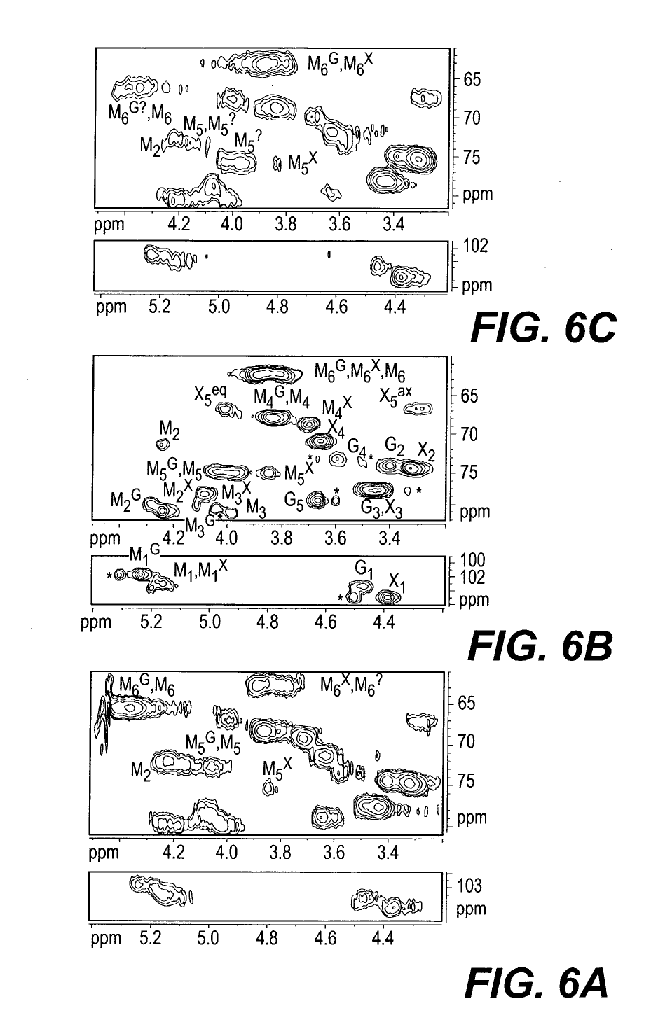

The structures of the isolated GXM were determined by NMR spectroscopy. Similar GXM compositions was found for the original strains JEC21 and JEC43, the mutants NE30 and NE31, and the reconstructed isolates NE30+pNE10-CAS1 and NE31+pNE10-CAS1. NMR data (see Table III, FIGS. 5 & 6) were compared with those of previously published de-O-acetylated GXMs from serotype D strains (Bacon et al., 1996; Skelton et al., 1991). They were consistent with a Chem1 chemotype (SRG M1: 70-80%; SRG M6: 20-30% (Cherniak et al., 1998).

In addition to resonance of the carbohydrate residues, intensive O-acetyl group signals were found at approximately 2.2 PPM for the original and reconstructed strains. Molar ratios of O-acetylation were determined from [¹H] NMR spectra by comparison of the acetyl resonances with those of the anomeric protons of mannose residues. These ratios varied between 0.63 (JEC43) and 0.58 (JEC21) acetyl groups per mannose residue in the original strains and 0.25 (NE30+pNE10-CAS1) and 0.24 (NE31+pNE10-CAS1) for the reconstructed strains. No acetylation was detected for the mutated strains (NE30 and NE31).

O-Acetylated residues were assigned using 2D NMR methods and comparison with known chemical shift effects for carbohydrate acetylation (Cherniak et al., 1988). Comparison of the chemical shifts of strains JEC21 and JEC43 with those of NE30 and NE31 indicated that no or only minor changes occurred for xylose and glucuronic acid residues, thereby indicating that they were not O-acetylated.

Table III shows that ¹H and ¹³C chemical shifts for some H-6 and C-6 of mannosyl residues in the original strain shifted by approximately +0.5 and +3 PPM, respectively, when compared to the mutant strain. Other substantial changes were observed for H-5/C-5 (+0.1/−3 PPM). These changes are consistent with 6-O-acetylation (Cherniak et al., 1988). Most of these changes were observed for the mannosyl residues without glycosylation in position 2 (Man) and for the mannosyl residue with a (1→2)-β-D-glucopyranosyluronic residue (Man^{GlcA}). Although it is difficult to specifically quantify 6-O-acetylation of the mannosyl residues because of partial signal overlapping, there are only minor changes in relative signal intensity (cross-peaks in HSQC spectra, FIG. 5) for the mannosyl residues with (1→2)-β-D-xylopryranosyl residue (Man^{Xyl}), indicating less or no 6-O-acetylation. These results are in agreement with two intense, resolved O-acetyl resonances in the [¹H] NMR spectra of the original strains at 2.18 and 2.20 ppm (FIG. 5).

GXM from the reconstructed strains (NE30+CAS1 and NE31+CAS1) were partially 6-O-acetylated but to a lesser extent than the original strains. Chemical shifts for all residues were identical to those of the original strain. However, relative cross-peak intensities in the HSQC spectra suggest less of 6-O-acetylation of the Man^{GlcA} residue. The NMR spectra indicate that 6-O-acetylation was mainly found in the Man residue, as is also supported by the presence of only one O-acetyl resonance in the [¹H] NMR spectra (2.20 ppm).

### EXAMPLE 6

Capsule Thickness Measurement

Capsule thickness of the 28 strains isolated by crossing after backcrossing the strain NE30 was determined as described under Materials and Methods. Genotype cas1-Δ strains (1.80±0.39) had a slightly thinner capsule than the genotype CAS1 strains (2.24±0.41 mm).

### EXAMPLE 7

Virulence Study

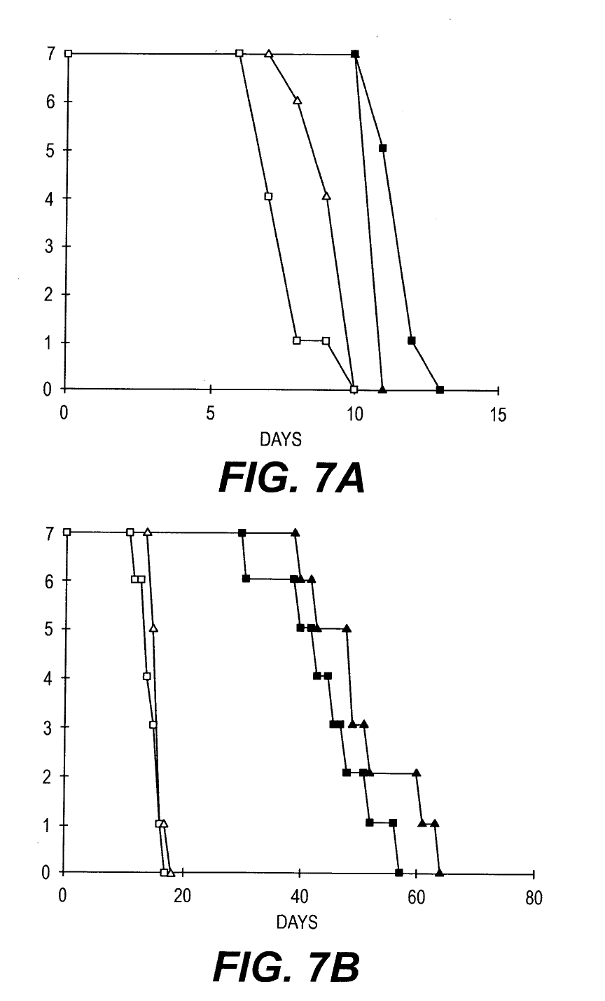

The virulence of 16 strains obtained after different crosses was tested in a murine model of disseminated infection. In two experiments, using an inoculum of 107 yeast cells (experiments 2 and 4), there there were only a slight difference although statistically significant (logrank test p<0.0001) between cas1-Δ strains and the original type ones. (see FIG. 7A). However, in two other ones experiments, using an inoculum of 10⁶ yeast cells (experiments 1 and 3), the cas1-Δ strains appeared clearly more virulent than the original strains. As shown in FIG. 7B, all mice infected with the cas1-Δ strains died between days 10 and 16 postinoculation, whereas all the mice infected with the original strains were still alive on day 30 (see FIG. 7B).

Discussion

In a recent review, Doering estimated that at least 12 transferases are required to synthesize the GXM and the GalXM of C. neoformans (Doering, 2000). Capsule-structure analysis clearly showed that the genotype cas1-Δ strains synthesized a GXM devoid of O-acetyl residues but that the glycosyl linkage between carbohydrate residues on the polysaccharide structure was not modified by CAS1 deletion. Moreover, the PropSearch analysis indicated that Cas1p belongs to the same protein family as different glycosyltransferases. This finding suggests that Cas1p might be an O-acetyltransferase. However, the amino-acid sequence of Cas1p does not share any homology with other putative O-acetyltransferases involved in bacterial polysaccharide synthesis (Bhasin et al., 1998; Franklin and Ohman, 1996; Reuber and Walker, 1993) or protein N-terminal acetylation (Polevoda et al., 1999). Actually, prediction of a function of a protein by analysis of its amino acids sequence may be sometime misleading and even though we have clearly demonstrated that Cas1p is an element of the biosynthetic pathway leading to the O-acetylation of the GXM.

No paralogue gene could be identified in the C. neoformans var. neoformans genome by Southern blotting using low stringency conditions or in the partial genome sequence available at Stanford University. This finding suggests that Cas1p might be also necessary for the GalXM acetylation. It also suggests that the strain from which CAS1 has been deleted is producing a completely de-O-acetylated capsule polysaccharide. On the other hand, we showed that at least one CAS1 homologue was present in the genomes of the four serotypes, a finding that is in good agreement with the presence of O-acetyl residues in all serotype GXM structures, even though their percentages vary according to the serotype (Turner and Cherniak, 1991).

Cas1p is an evolutionary well-conserved protein from sequence and structure points of view. In mammals, O-acetylation is one of most common modifications that occur on sialic acids. The presence of O-acetylated sialic acid has selective and widespread distribution and is developmentally regulated (Klein et al., 1994; Varki, 1992). Even though the biological function of these residues is still unknown, it has been demonstrated that numerous cancers are associated with a deregulation of the sialic acids O-acetylation (Brockenhausen and Kuhns, 1997; Klein and Roussel, 1998; Pal et al., 2000) and that O-acetylation of plant cell wall polysaccharides exists (Pauly and Scheller, 2000). Interestingly, we were unable to identify any homologue in the complete S. cerevisiae genome or in other partially available ascomycete yeast genomes. Similar results were obtained with two other genes cloned from C. neoformans and involved in GXM biosynthesis that have clear homologues in the genomes of various higher eukaryotes but none in the ascomycete genomes (G. Janbon, unpublished data).

Although GXM structures from the four serotypes have been thoroughly analyzed from a chemical point of view, the position of the O-acetyl group was largely unknown. We showed here that O-acetylation occurs on C-6 of either Man or Man^{GlcA} residues. CAS1 deletion led to complete de-O-acetylation of the GXM showing that Cas1p is necessary for O-acetylation of the mannosyl backbone.

This invention also provide some clues about the cascade of events leading to the complete GXM structure. First, no O-acetylation of Man^{Xyl} residues was observed. Second, no or poor O-acetylation of Man^{GlcA} was observed in the reconstructed strains. Third, xylosylation and glucuronosylation of the mannosyl residues was not affected by the mutation. The most likely explanation is that the GlcA and Xyl residues are transferred to the mannose backbone before the O-acetyl residues. By analogy with the biosynthesis of bacterial alginate the GXM acetylation would be the last step in their biosynthesis and could be linked with polysaccharide secretion (Gacesa, 1998).

Reintroduction of the CAS1 gene on a multicopy plasmid did not fully restore the amount of O-acetylation. The reconstructed strains harbored fewer O-acetyl residues, and they were mostly present on unglycosylated Man residues. The simplest explanation is that the prolonged growth of the strains in induction medium before GXM purification led to a partial loss of the plasmid and, subsequently to less Cas1p per cell. Our results also suggest that the Man residues are preferentially O-acetylated compared to Man^{GlcA} residues and that variation of the Cas1p cell content can lead to changes in GXM structure. Our immunoblot results also showed some differences among the E1 reactivities with the original strain, the reconstructed one and the original strain transformed with a plasmid containing CAS1. These results are in good agreement with the hypothesis of a lower Cas1p cell concentration when CAS1 is on a plasmid than when it is at its natural chromosomal location.

CAS1 isolated from C. neoformans var. grubii was able to complement the mutation of the C. neoformans var. neoformans strain, thereby suggesting that the small divergence between the two Cas1p sequences was not responsible for the antigenic specificity of the two serotypes. However, the E1 reactivities with cas1-□ strains reconstructed with CAS1 either from serotype A or serotype D differed from that of the original strain. This finding raises two hypotheses: (1) serotype A Cas1p is able to O-acetylate Man and Man^{GlcA} residues in a Chem1-chemotype context but, less protein is synthesized in reconstructed strains than in the original one; (2) serotype A Cas1p acetylates only one position on the GXM.

The role of external sugar O-acetylation has been previously studied for other microorganisms. The O-acetylation of the capsule has been proven to be essential for the virulence of Staphylococcus aureus (Bhasin et al., 1998) but, for Salmonella typhimurium, de-O-acetylation of its lipopolysacharide has no effect on the virulence of the strains (Michetti et al., 1992; Slauch et al., 1995). Comparison of the virulence of cas1-□ and original strains showed that the former with a non-O-acetylated capsule were, at least in two experiments more virulent than the latter in a murine model. This result represents the first demonstration that the capsule structure can influence the pathophysiology of cryptococcosis. However, it must be kept in mind that, even though this murine model of cryptococcosis which mimics disseminated infection in humans has been previously validated (Lortholary et al., 1999a; Lortholary et al., 1999b), it does not mean it is relevant for other clinical situations. For example, this model does not take into account the most common route of infestation, i.e. inhalation, nor the dormant phase of the infection (Garcia-Hermoso et al., 1999). The higher virulence of cas1-Δ strains is difficult to explain. As postulated above, the changed structural composition might influence many in vitro parameters that could interfere with the course of the infection and therefore could be responsible for the apparent modification of in vivo virulence. Cleare and colleagues recently reported the attenuated virulence of a spontaneous variant strain that synthesized a poorly O-acetylated GXM and suggested a causal relationship between the two phenotypes (Cleare et al., 1999). This finding contrasts strongly with our results probably because they did not compare isogenic strains in their virulence assay. The de-O-acetylation of GXM was associated in our strains with a thinner capsule which is in good agreement with the previously reported reduction of capsule thickness after its chemical de-O-acetylation (Young and Kozel, 1993). Our results also confirms that capsule thickness does not directly influence strain virulence.

Further studies are needed to explain the influence of each GXM structural element on the pathophysiology of cryptococcosis. In this context, it will be necessary to identify other genes involved in the capsule-structure biosynthesis of C. neoformans, which represents a very exciting area of research.

The implication for this invention are widespread.a cdna encoding hsCas1p has been isolated and is disclosed in SEQ ID NO:2. This discovery of the cdna encoding hsCas1p proteins enbles construction of expression vector comprising nucleic acid sequences encoding hsCas1p polypeptides; host cells transfected or transformed with the expression vectors; biologically active hsCas1p polypeptides and hsCas1p polypeptides as isolated or purified proteins; and antibodies immunoreactive with hsCas1p polypeptides. In addition, the hsCas1p polypeptides enable the design of assays to detect inhibitors of hsCas1p protein activity.

### REFERENCES

1. Franzot, S. P., Salkin, I. F., and Casadevall, A. (1999) J. Clin. Microbiol. 37, 838-840.

2. Kwon-Chung, K. J., and Bennett, J. E. (1984) Am. J. Epidemiol. 120, 123-130.

3. Casadevall, A., and Perfect, J. R. (1998) Cryptococcus neoformans, American Society for Microbiology Press, Washington, D.C.

4. Cherniak, R., and Sundstrom, J. B. (1994) Infect. Immun. 62, 1507-1512.

5. Cherniak, R., Valafar, H., Morris, L. C., and Valafar, F. (1998) Clin. Diagn. Lab. Immunol. 5, 146-159.

6. Buchanan, K. L., and Murphy, J. W. (1998) Emerg. Infect. Dis. 4, 71-83.

7. Bennett, J. E., and Hasenclever, H. F. (1965) J. Immunol. 94, 916-920.

8. Chang, Y. C., and Kwon-Chung, K. J. (1994) Mol. Cell. Biol. 14, 4912-4919.

9. Chang, Y. C., Penoyer, L. A., and Kwon-Chung, K. J. (1996) Infect. Immun. 64, 1977-1983.

10. Chang, Y. C., and Kwon-Chung, K. J. (1998) Infect. Immun. 66, 2230-2236.

11. Chang, Y. C., and Kwon-Chung, K. J. (1999) J. Bacteriol. 181, 5636-5643.

12. Doering, T. L. (2000) Trends Microbiol. 8, 547-553.

13. Jacobson, E. S., and Tingler, M. J. (1994) J. Med. Vet. Mycol. 32, 401-404.

14. Jacobson, E. S., Ayers, D. J., Harrel, A. C., and Nicholas, C. C. (1982) J. Bacteriol. 150, 1292-1296.

15. Jacobson, E. S., and Payne, W. R. (1982) J. Bacteriol. 152, 932-934.

16. Jacobson, E. S. (1987) J. Med. Vet. Mycol. 25, 131-135.

17. White, C. W., Cherniak, R., and Jacobson, E. S. (1990) J. Med. Vet. Mycol. 28, 289-301.

18. Doering, T. L. (1999) J. Bacteriol. 181, 5482-5488.

19. Dromer, F., Mathoulin, S., Dupont, B., Letenneur, L., Ronin, O., and the French Cryptococcosis Study Group. (1996) Clin. Infect. Dis. 23, 91-96.

20. Small, J. M., and Mitchell, T. G. (1989) Infect. Immun. 57, 3751-3756.

21. Lipovsky, M. M., Gekker, G., Hu, S., Ehrlich, L. C., Hoepelman, I. M., and Peterson, P. K. (1998) J. Infect. Dis. 177, 260-263.

22. Chaka, W., Verheul, A. F. M., Vaishnav, V. V., Cherniak, R., Scharringa, J., Verhoef, J., Snippe, H., and Hoepelman, I. M. (1997) Infect. Immun. 65, 272-278.

23. Washburn, R. G., Bryant-Valera, B. J., Julian, N.C., and Bennett, J. E. (1991) Mol. Immunol. 28, 465-470.

24. Young, B. J., and Kozel, T. (1993) Infect. Immun. 61, 2966-2972.

25. Sherman, F. (1992) in Guide to yeast genetics and molecular biology (Guthrie, C., and Fink, G. R., eds) Vol. 194, pp. 3-21, Academic Press, Inc., San Diego, Calif.

26. Granger, D. L., Perfect, J. R., and Durack, D. T. (1985) J. Clin. Invest. 76, 508-516.

27. Hanahan, D. (1983) J. Mol. Biol. 166, 557-560.

28. Dromer, F., Salamero, J., Contrepois, A., Carbon, C., and Yeni, P. (1987) Infect. Immun. 55, 742-748.

29. Ikeda, R., Nishimura, S., Nishikawa, A., and Shinoda, T. (1996) Clin. Diagn. Lab. Immunol. 3, 89-92.

30. Casadevall, A., and Scharff, M. D. (1991) J. Exp. Med. 174, 151-160.

31. Garcia-Hermoso, D., Janbon, G., and Dromer, F. (1999) J. Clin. Microbiol. 37, 3204-3209.

32. Sambrook, J., Fritsch, E. F., and Maniatis, T. (1989) Molecular cloning. A Laboratory manual, 2nd Ed., Cold Spring Harbor Laboratory, Cold Spring Harbor, N.Y.

33. Devereux, J. P., Haeberli, P., and Smithies, O. (1984) Nucleic Acid Res. 12, 387-395.

34. Sonnhammer, E. L. L., von Heijne, G., and Krogh, A. (1998) in Proc. of Sixth Int. Conf. on Intelligent Systems for Molecular Biology (Glasgow, J., Littlejohn, T., Major, F., Lathrop, R., Sankoff, D., and Sensen, C., eds), pp. 175-182, AAAI Press, Menlo Park, Calif.

35. Hobohm, U., and Sander, C. (1995) J. Mol. Biol. 251, 390-399.

36. Cherniak, R., Morris, L. C., Anderson, B. C., and Meyer, S. A. (1991) Infect. Immun. 59, 59-64.

37. Dromer, F., Guého, E., Ronin, O., and Dupont, B. (1993) J. Clin. Microbiol. 31, 359-363.

38. Kwon-Chung, K. J., Bennett, J. E., and Rhodes, J. C. (1982) Antonie van Leeuwenhoek 48, 25-38.3

39. Edman, J. C., and Kwon-Chung, K. J. (1990) Mol. Cell. Biol. 10, 4538-4544.

40. Sudarshan, S., Davidson, R. C., Heitman, J., and Alspaugh, J. A. (1999) Fungal Genet. Biol. 27, 36-48.

41. Wickes, B. L., Moore, T. D. E., and Kwon-Chung, K. J. (1994) Microbiology 140, 543-550.

42. Rivera, J., Feldmesser, M., Cammer, M., and Casadevall, A. (1998) Infect. Immun. 66, 5027-5030.

43. Lortholary, O., Improvisi, L., Nicolas, M., Provost, F., Dupont, B., and Dromer, F. (1999) Med. Mycol. 37, 169-174.

44. Walker, J. E., Saraste, M., Runswick, M. J., and Gay, N.J. (1982) EMBO J. 1, 945-951.

45. Strahl-Bolsinger, S., Gentzsch, M., and Tanner, W. (1999) Biochim. Biophys. Acta 1426, 297-307.

46. Skelton, M. A., Cherniak, R., Poppe, L., and van Halbeek, H. (1991) Magn. Res. Chem. 29, 786-793.

47. Bacon, B. E., Cherniak, R., Kwon-Chung, K. J., and Jacobson, E. S. (1996) Carbohydr. Res. 283, 95-110.

48. Cherniak, R., Jones, R. G., and Reiss, E. (1988) Carbohydr. Res. 172, 113-138.

49. Bhasin, N., Albus, A., Michon, F., Livolsi, P. L., Park, J. S., and Lee, J. C. (1998) Mol. Microbiol. 27, 9-21.

50. Reuber, T. L., and Walker, G. C. (1993) J. Bacteriol. 175, 3653-3655.

51. Franklin, M. J., and Ohman, D. E. (1996) J. Bacteriol. 178, 2186-2195.

52. Polevoda, B., Norbeck, J., Takakura, J., Blomberg, A., and Sherman, F. (1999) EMBO J. 18, 6155-6168.

53. Wiggins, C. A. R., and Munro, S. (1998) Proc. Natl. Acad. Sci. USA 95, 7945-7950.

54. Kapitonov, D., and Yu, R. K. (1999) Glycobiology 9, 961-978.

55. Turner, S. H., and Cherniak, R. (1991) in Fungal Cell Wall and Immune Response (Latgé, J. P., and Boucias, D., eds) Vol. H 53, pp. 123-142, NATO ASI Series, Heidelberg.

56. Klien, A., Krishna, M., Varki, N. L., and Varki, A. (1994) Proc. Natl. Acad. Sci. USA 91, 7782-7786.

57. Varki, A. (1992) Glycobiology 2, 25-40.

58. Brockenhausen, I., and Kuhns, W. (1997) in Glycoproteins and human disease (Brockenhausen, I., and Kuhns, W., eds), pp. 176-193, R. G. Lands Compagny.

59. Pal, S., Chatterjee, M., Bhattacharya, D. K., Bandhyopadhyay, S., and Mandal, C. (2000) Glycobiology 10, 539-549.

60. Klein, A., and Roussel, P. (1998) Biochimie 80, 49-57.

61. Pauly, M., and Scheller, H. V. (2000) Planta 210, 659-667.

62. Gacesa, P. (1998) Microbiology 144, 1133-1143.

63. Michetti, P., Mahan, M. J., Slauch, J. M., Mekelanos, J. J., and Neutra, M. R. (1992) Infect. Immun. 60, 1786-1792.

64. Slauch, J. M., Mahan, M. J., Michetti, P., Neutra, M., and Mekalanos, J. J. (1995) Infect. Immun. 63, 437-441.

65. Spath, G. F., Epstein, L., Leader, B., Singer, S. M., Avila, H. A., Turco, S. J., and Beverley, S. M. (2000) Proc. Natl. Acad. Sci. USA 97, 9258-9263.

66. Ilg, T. (2000) EMBO J. 19, 1953-1962.

67. Lortholary, O., Improvisi, L., Rayhane, N., Gray, F., Fitting, C., Cavaillon, J. M., and Dromer, F. (1999) Infect. Immun. 67, 6314-6320.

68. Cleare, W., Cherniak, R., and Casadevall, A. (1999) Infect. Immun. 67, 3096-3107.

69. Moore, T. D. E., and Edman, J. C. (1993) Mol. Cell. Biol. 13, 1962-1970.

70. Wickes, B. L., and Edman, J. C. (1995) Mol. Microbiol. 16, 1099-1109.

71. Kwon-Chung, K. J., Edman, J. C., and Wickes, B. L. (1992) Infect. Immun. 60, 602-605.

72. Loubens, I., Debardieux, L., Bohin, A., Lacroix, J. M., and Bohin, J. P. (1993) Mol. Microbiol. 10, 329-340.

73. Sofia, H. J., Burland, V., Daniels, D. L., Plunkett, G. r., and Battner, F. R. (1994) Nucleic Acid Res. 22, 2576-2586.

74. Lussier, M., Gentzsch, M., Sdicu, A. M., Bussey, H., and Tanner, W. (1995) Journal of Biological Chemistry 270, 2770-2775.

75. Garvey, K. J., Oberste, M. S., Elser, J. E., Braun, M. J., and Gonda, M. A. (1990) Virology 175, 391-409.

76. Mukhopadhyay, P., Williams, J., and Mills, D. (1988) J. Bacteriol. 170, 5479-5488.

77. Simon, D. B., Nelson-Williams, C., Bia, M. J., Ellison, D., Karet, E. E., Molina, A. M., Vaara, I., Iwata, F., Cushner, H. M., Koolen, M., Gainza, F. J., Gitelman, H. J., and Lifton, R. P. (1996) Nat. Genet. 12, 24-30.

78. Sato, S., Suzuki, H., Widyastuti, U., Hotta, Y., and Tabata, S. (1994) Curr. Genet. 26, 31-37.

79. Immervoll, I., Gentzsch, M., and Tanner, W. (1995) Yeast 11, 1345-1351.

80. Strahl-Bolsinger, S., Immervol, T., Deutzmann, R., and Tanner, W. (1993) Proc. Natl. Acad. Sci. USA 90, 8164-8168.

81. Standal, R., Iversen, T. G., Coucheron, D. H., Fjaervik, E., and Blatny, J. M. (1994) J. Bacteriol. 176, 665-672.

82. Wong, H. C., Fear, A. L., Calhoom, R. D., Eichinger, G. H., Mayer, R., Amikan, D., Benziman, M., Gelfand, D. H., Meade, J. H., Emerick, A. W., Bruner, R., Ben-Bassat, A., and Tal, R. (1990) Proc. Natl. Acad. Sci. USA 87, 8130-8134.

Footnotes.

The sequence data have been submitted to the DDBJ/EMBL/GenBankdatabases under accession numbers AF355592, AF355593 and AF355594, respectively.

What is claimed is:

1. A purified polynucleotide of SEQ ID NO: 1.

2. A purified polynucleotide encoding a polypeptide of SEQ ID NO:2.

3. A purified polynucleotide according to claim 2, which encodes hsCas1p polypeptide.

4. A purified polynucleotide according to claims 1, 2 or 3, which encodes an allelic variant of hsCas1p polypeptide.

5. A purified polynucleotide according to claims 1, 2 or 3, which encodes a homolog of hsCas1p polypeptide.

6. A purified polynucleotide that hybridizes to either strand of a denatured, double-stranded DNA comprising the polynucleotide of any one of claims 1 to 5 under conditions of high stringency.

7. A purified polynucleotide sequence derived by in vitro mutagenesis from SEQ ID NO1.

8. A purified polynucleotide sequence degenerate from SEQ ID NO:1 as the result of genetic code.

9. A nucleotidic probe comprising at least 8 amino acids of the polynucleotide sequence of SEQ ID NO: 1.

10. A nucleotidic primer comprising at least 8 amino acids of the polynucleotide sequence of SEQ ID NO: 1.

11. A primer comprising the nucleotidic sequence of SEQ ID NO: 5.

12. A primer comprising the nucleotidic sequence of SEQ ID NO: 6.

13. A primer comprising the nucleotidic sequence of SEQ ID NO: 7.

14. A primer comprising the nucleotidic sequence of SEQ ID NO: 8.

15. A primer comprising the nucleotidic sequence of SEQ ID NO: 9.

16. A primer comprising the nucleotidic sequence of SEQ ID NO: 10.

17. A purified polypeptide encoded by a polynucleotide selected from the group consisting of the purified polynucleotides of claims 1, 2, 3, 4, 5, 6, 7 and 8.

18. A purified polypeptide according to claim 17 having a molecular weight of approximately 108 kDa.

19. A purified polypeptide according to claim 18 in post translationnally modified form or not.

20. A purified polypeptide according to claims 17, 18 or 19 which has a glycosyltransferase activity.

21. A polynucleotide fragment comprising at least 10 contiguous nucleotides that hybridizes under high stringency conditions with a sequence according to claim 1 or the complementary fragment thereof.

22. A purified polypeptide or a peptide fragment having at least 10 amino acids, which is recognized by antibodies directed against a peptide encoded by a polynucleotide sequence corresponding to the polynucleotide sequence of claim 1.

23. A recombinant DNA molecule comprising at least one nucleotide sequence according to claim 1 under the control of regulatory elements that regulates the expression of the glycosyltransferase in a host.

24. A chimeric protein comprising at least a polypeptide according to any one of claims 17 to 22.

25. A recombinant vector that directs the expression of a nucleic acid molecule selected from the group consisting of the purified polynucleotide of claims 1, 2, 3, 4, 5, 6, 7 and 8.

26. A plasmid pNE8 deposited at the C.N.C.M. on May 10, 2001 under the accession number I-2666.

27. A plasmid pPNE95 deposited at the C.N.C.M. on May 10, 2001 under the accession number I-2667.

28. A recombinant cell host comprising SEQ ID NO:1 or the recombinant DNA molecule of claim 23.

29. An isolated host cell transfected or transduced with a recombinant vector according to claims 25 to 27.

30. Purified antibodies that bind to a polypeptide according to claim 17.

31. Purified antibodies according to claim 22, wherein said antibodies are monoclonal antibodies.

32. Purified antibodies that bind to a polypeptide according to claims 18, 19, 20.

33. Purified antibodies according to claim 24, wherein said antibodies are monoclonal antibodies.

34. A method for the production of hsCas1p polypeptide comprising culturing a host cell according to claims 28 or 29 under conditions promoting expression, and recovering the polypeptide from the host cell or the culture medium.

35. The method of claim 34, wherein the host cell is selected from the group consisting of bacterial cells, parasite cells and eukariotic cells.

36. An isolated immunological complex comprising a hsCas1p polypeptide and an antibody according to claims 30 to 34.

37. A composition comprising at least one polypeptide encoded by hsCAS1 polynucleotide sequence.

38. A composition comprising at least one nucleotide sequence according to claim 1 that encodes a glycosyltransferase.

39. A composition comprising at least one polynucleotide that is selected from the group consisting of a polynucleotide sequence according to claims 1, 2, 3, 4, 5, 6, 7 or 8.

40. A diagnostic method for the predictive prognosis of cancer in a patient comprising the following steps:
a) Preparing two tissue samples, the first tissue sample being derived from an organ suspected to be tumorous and a second tissue sample being derived from a healthy patient;
b) Optionnally making the RNA contained in the cells of the tissue sample of step a) available to hybridization;
c) Amplifying the RNA with at least one primer according to the invention,
d) Measuring the level of transcription of the DNA sequence corresponding to the hsCAS1 gene contained in genomic DNA of said tissue samples,
e) Detecting the deregulation of transcription that have occured in the biosynthesis of o-Acetyltransferase in the sample derived from the patient suspected to be tumorous by comparing the resulting product of step d) derived respectively from the first and the second tissue sample.

41. A diagnostic method for the predictive prognosis of colorectal cancer in a patient comprising the following steps:
a) Preparing two tissue samples from a patient, the first tissue sample being derived from an organ different than the colon and a second tissue sample being derived from the colon of said patient;
b) Optionnally making the RNA contained in the cells of the tissue sample of step a) available to hybridization;
c) Amplifying the RNA with at least one primer according to the invention,
d) Measuring the level of transcription of the DNA sequence corresponding to the hsCAS1 gene contained in genomic DNA of said tissue samples,
e) Detecting the deregulation of transcription that have occured in the biosynthesis of o-Acetyltransferase in the sample derived from the colon by comparing the resulting product of step d) derived respectively from the first and the second tissue sample.

42. The diagnostic method according to claim 40 or 42, wherein the measure of the level of transcription of the DNA sequence at step d) is done by the technique of quantitative RT-PCR.

43. A kit containing for the diagnostic of cancer containg antibodies according to any one of claims 30 to 33.

44. A purified polynucleotide sequence of SEQ ID NO: 3.

45. A purified polynucleotide sequence encoding a polypeptide of SEQ ID NO:4.

46. A purified polynucleotide sequence according to claim 44, which encodes cnCas1p polypeptide.

47. A purified polynucleotide sequence according to claim 46, which encodes an allelic variant of cnCas1p polypeptide.

48. A purified polynucleotide sequence according to claim 46 which encodes a homolog of cnCas1p polypeptide.

49. A purified polynucleotide sequence that hybridizes to either strand of a denatured, double-stranded DNA comprising the polynucleotide of any one of claims to 44 to 48 under conditions of high stringency.

50. The purified polynucleotide sequence derived by in vitro mutagenesis from SEQ ID NO:3.

51. A purified polynucleotide sequence degenerate from SEQ ID NO:3 as the result of genetic code.

52. A polynucleotide molecule encoding a glycosyltransferase that hybridizes to either strand of a denatured, double-stranded DNA comprising the polynucleotide of any one of claims 44 to 48 under conditions of high stringency.

53. A purified polypeptide having the amino acid sequence of SEQ ID NO 4.

54. A purified polypeptide molecule encoded by a polynucleotide sequence selected from the group consisting of the purified polynucleotides sequences acoording to any one of claims 44 to 51.

55. A purified polypeptide according to claim 53 or 54 having a molecular weight of approximately 108 kDa.

56. A purified polypeptide according to claim 53 or 54 in post translationnally modified form or not.

57. A purified polypeptide according to claim 53 or 54 which has a glycosyltransferase activity.

58. A polynucleotide fragment comprising at least 10 contiguous nucleotides that hybridizes under high stringency conditions with a sequence according to claim 44 or the complementary fragment thereof.

59. A nucleotidic probe comprising at least 8 amino acids of the polynucleotide sequence of SEQ ID NO: 3.

60. A nucleotidic primer comprising at least 8 amino acids of the polynucleotide sequence of SEQ ID NO: 3.

61. A purified polypeptide or a peptide fragment having at least 10 amino acids, which is recognized by antibodies directed against a peptide encoded by a polynucleotide sequence corresponding to the polynucleotide sequence of claim 44.

62. A recombinant DNA molecule comprising at least one polynucleotide sequence according to claim 44 under the control of regulatory elements that regulates the expression of the glycosyltransferase in a host.

63. A chimeric protein comprising at least one polypeptide according to claim 53 or 54.

64. A recombinant vector that directs the expression of a nucleic acid molecule comprising the purified polynucleotide sequence of claims 44 to 52.

65. A recombinant cell host comprising SEQ ID NO:3 or the recombinant molecule of claim 62.

66. An isolated host cell transfected or transduced with a recombinant vector according to claims 64.

67. Purified antibodies that bind to a polypeptide according to claim 53.

68. Purified antibodies according to claim 67, wherein said antibodies are monoclonal antibodies.

69. Purified antibodies that bind to a polypeptide according to 54.

70. Purified antibodies according to claim 69, wherein said antibodies are monoclonal antibodies.

71. A method for the production of cnCas1p polypeptide comprising culturing a host cell according to claims 65 or 66 under conditions promoting expression, and recovering the polypeptide from the host cell or the culture medium.

72. The method of claim 71, wherein the host cell is selected from the group consisting of bacterial cells, parasite cells and eukariotic cells.

73. An isolated immunological complex comprising a hCas1p polypeptide and an antibody according to any one of claim 68 to 70.

74. A composition comprising at least one polypeptide encoded by cnCAS1 polynucleotide sequence.

75. A composition comprising at least one polynucleotide sequence according to claim 44 that encodes a glycosyltransferase.

76. A composition comprising at least one polynucleotide which is selected from the group consisting of a polynucleotide sequence according to 44 to 51.

77. A purified polynucleotide sequence comprising the SEQ ID NO:11.
